(19) United States
(12) Patent Application Publication
(10) Pub. No.: US 2024/0390190 A1
(43) Pub. Date: Nov. 28, 2024

(54) WOUND DRESSING

(71) Applicant: AROA BIOSURGERY LIMITED, Auckland (NZ)

(72) Inventors: Brian Roderick WARD, Pukekohe (NZ); Isaac Tristram Tane MASON, Auckland (NZ); Hamish Joshua ROSE, Auckland (NZ); Samuel Barry TURNER, Auckland (NZ); Sarah Frances WILLIAMSON, Auckland (NZ); Nigel TRINIDADE, Auckland (NZ); Sean James GORMAN, Auckland (NZ); Dorrin ASEFI, Auckland (NZ); Henry David CHITTOCK, Auckland (NZ)

(21) Appl. No.: 18/038,356

(22) PCT Filed: Nov. 24, 2021
(86) PCT No.: PCT/NZ2021/050208

§ 371 Date: May 23, 2023

Related U.S. Application Data
(60) Provisional application No. 63/217,948, filed on Jul. 2, 2021.
(60) Provisional application No. 63/117,995, filed on Nov. 24, 2020.
(60) Provisional application No. 63/280,787, filed on Nov. 18, 2021.

(51) Int. Cl. A61F 13/05; A61M 1/00

(52) U.S. Cl. CPC A61F 13/05; A61M 1/912; A61M 1/92; A61M 1/966

(57) ABSTRACT

A wound interface device for use in negative pressure wound therapy. The device includes a flexible body having a plurality of spacers, the spacers configured to define a therapy space between a wound surface and a wound facing surface of the device body. The device also includes an inlet for the instillation of fluids to a wound site and an outlet for the removal of fluids from the therapy space.

### CROSS-REFERENCE TO RELATED APPLICATIONS

This application is a 35 U.S.C. § 371 national stage filing of International Application No. PCT/NZ2021/050208, filed Nov. 24, 2021, which claims priority to U.S. provisional application No. 63/117,995, filed Nov. 24, 2020, U.S. provisional application No. 63/217,948, filed Jul. 2, 2021, and U.S. provisional application No. 63/280,787, filed Nov. 18, 2021. The contents of the aforementioned applications are hereby incorporated by reference in their entirety.

### FIELD OF THE INVENTION

This invention relates to a wound dressing and a device for a wound dressing, in particular a dressing for the application of negative pressure and/or for the instillation of treatment fluids to a wound.

### BACKGROUND

The technique of applying negative pressure to augment the healing of soft tissues has been utilised for many years with the core principle of the therapy remaining largely unchanged.

In the context of treating open wounds, negative pressure wound therapy (NPWT) typically involves the placement of porous materials such as an open-cell foam, reticulated foam or gauze onto the wound site, sealing the wound cavity with an occlusive layer and applying a negative pressure to the sealed wound environment. The clinical efficacy of this treatment is well supported in areas such as for acute and chronic wounds, which have demonstrated accelerated formation of granulation tissue in open wounds in response to the treatment. The negative pressure is applied with the intent to remove excess fluid from the wound site, since excess fluid is problematic for wound healing, increases the risk of infection, oedema and may lead to biofilm formation and thereby subsequent stalled healing.

While the open architecture of the porous wound contacting layer allows for the effective application of pressure to the wound, a shortcoming with present NPWT dressing constructs is the susceptibility for healing granulation tissue to grow into the porous wound contacting layer. This results in trauma to the newly formed tissue when the foam layer is removed. To prevent or minimise tissue in-growth it is necessary to regularly change the dressing which requires additional time and expense and can induce acute trauma to the peri-wound or intact skin area around the wound further compounding the overall treatment time.

Further, it is desirable to maintain a level of moisture at open wound sites to support healing. In particular, foam-based dressings are generally unsuitable for use in wounds containing exposed tendons and ligaments, particularly when combined with the application of negative pressure which may cause the wound to dry out and hinder healing.

In NPWT, negative pressure is provided to the wound treatment space by a conduit leading from the wound site to a vacuum source such as a vacuum pump. The vacuum pump and conduit can be susceptible to becoming blocked by coagulated blood, fibrin, adipose tissue, lose tissue debris and wound exudate.

Some wound treatment systems involve delivering a treatment fluid to the wound to assist with healing. FIGS. 1 to 3 illustrate some prior art systems 101. The treatment fluid is typically delivered to the wound under a positive pressure (above atmospheric pressure) via a fluid supply conduit 151. The positive pressure ensures complete saturation of the wound site with the treatment fluid but results in the wound site remaining at an ambient or positive pressure level. A negative pressure may be applied after the application of the treatment fluid to reduce the free volume of the sealed wound environment and draw the treatment fluid and exudate away from the wound site via a fluid removal conduit 153. The positive pressure applied to the wound treatment site can have unintended consequences such as inducing a leak from the wound dressing, typically between the peri-wound and a cover dressing.

It is an object of at least preferred embodiments of the present invention to address one or more of the abovementioned disadvantages and/or to at least provide the public with a useful alternative.

In this specification where reference has been made to patent specifications, other external documents, or other sources of information, this is generally to provide a context for discussing features of the invention. Unless specifically stated otherwise, reference to such external documents or sources of information is not to be construed as an admission that such documents or such sources of information, in any jurisdiction, are prior art or form part of the common general knowledge in the art.

### SUMMARY OF THE INVENTION

According to a first aspect, the invention described herein broadly consists in a wound interface device for use in negative pressure wound therapy, the device comprising: a flexible body having a plurality of spacers, the spacers configured to define a therapy space between a wound surface and a wound facing surface of the device body; an inlet for the instillation of fluids to a wound site; and an outlet for the removal of fluids from the therapy space.

In an embodiment, the inlet is configured to deliver fluid to an area adjacent a first end of the device, and the outlet is configured to remove fluid from an area adjacent to a second, opposite, end of the device.

In an embodiment, the spacers define multiplicity of channels between the first and second ends of the device.

In an embodiment, the channels are shaped to direct fluids laterally across the wound surface between the first and second ends of the device.

In an embodiment, the channels are shaped in bridges, corrugations, or chevrons.

In an embodiment, the spacers are shaped and distributed to prevent collapse of the spacers and/or of the therapy space under the application of negative pressure to the therapy space.

In an embodiment, the spacers are bulbous or generally hemispherical. The spacers may include an undercut to define a deflection point of the spacer.

The walls of each spacer may be uniform or may vary at different parts of the spacer. A thinner wall region may be utilised to create an undercut region to define a deflection point of the spacer.

In an embodiment, the spacers comprise shaped projections.

In an embodiment, the spacers each comprise a convex wound contacting surface. In alternative embodiments, the spacers may comprise a concave wound facing surface.

In an embodiment, the spacers are spaced to prevent extracellular matrix (ECM) or extracellular matrix (ECM) material from lifting.

In an embodiment, the body comprises a compliant, flexible member that flexes to conform to the contours of various wound surfaces.

In an embodiment, the wound interface device is trimmable to fit within the perimeter of various wounds. In some embodiments, the body may comprise perforations between spacers to allow tearing of the device to customise its shape.

In an embodiment, the wound flexible member is compliant. The flexible member has a wall thickness.

In an embodiment, the inlet comprises an inlet port for coupling to a fluid source; and an inlet channel in fluid communication with the inlet port, configured to deliver fluid to a first end of the device.

In an embodiment, the outlet comprises an outlet port for coupling to a negative pressure source; and an outlet channel in fluid communication with the outlet port, configured to remove fluid from a second end of the device.

In an embodiment, the inlet channel extends in a first direction, and the outlet channel extends in a second direction that is perpendicular to the first direction. For example, the outlet channel extends in a transverse direction of the device. The inlet channel may extend in a longitudinal direction of the device.

In an embodiment, the inlet and outlet ports are provided on a moulded member extending from an opposite side of the device body to the wound facing side.

In an embodiment, the inlet ports are longitudinal to the outlet ports. In another embodiment, the inlet ports are transverse to the outlet ports.

In an embodiment, the outlet channel comprises an elongate slot positioned to allow the ingress of fluids from the therapy space into the outlet channel.

In an embodiment, the slot extends in a transverse direction of the device.

In an embodiment, the outlet channel comprises a plurality of apertures to allow the ingress of fluids from the therapy space into the outlet channel.

The outlet channel apertures may comprise slits that are substantially perpendicular to a plane of the body of the device.

In an embodiment, the wound interface device applies pressure across the width of wound.

The width of the slot may prevent blocking matter from entering the outlet port.

In an embodiment, the body is formed from an elastomeric material.

In an embodiment, the body is formed from silicone.

In an embodiment, the elastomeric material is a thermoplastic.

In an embodiment, the elastomeric material is provided with a coating.

In an embodiment, the body is liquid and air impermeable.

In an embodiment, the body has no apertures through it.

According to a second aspect, the invention described herein broadly consists in a wound dressing for applying negative pressure to a wound, the dressing comprising: a wound interface device according to the first aspect of the invention, and a liquid impermeable occlusive outer layer.

In an embodiment, the wound dressing further comprises a bioresorbable layer for placing between the wound interface device and the wound surface.

In an embodiment, the bioresorbable layer comprises a plurality of apertures to enable fluid flow from the wound to the porting layer.

In an embodiment, the apertures or slits are substantially X-shaped, Y-shaped, C-shaped, U-shaped, or V-shaped.

In an embodiment, the bioresorbable layer comprises a multiplicity of small pieces of bioresorbable material.

In an embodiment, the bioresorbable sheets layer comprises extracellular matrix (ECM).

In an embodiment, the wound dressing comprises an inlet port and an outlet port, and comprising an inlet conduit for coupling to the inlet port for the supply of fluids to the wound and an outlet conduit for the application of negative pressure to the wound.

In an embodiment, the inlet and outlet conduits are provided by a dual lumen conduit.

In an embodiment, the dual lumen conduit comprises a primary conduit to couple to the outlet to apply a negative pressure to the dressing and a secondary conduit to couple to the inlet for introducing fluid to the dressing and/or for facilitating pressure measurement.

In an embodiment, the occlusive layer comprises a polyurethane sheet comprising an adhesive surface.

In an embodiment, the wound dressing comprises a mouldable adhesive seal for surrounding a wound, wherein the seal comprises butyl rubber, a filler, and a tackifying resin.

In an embodiment, the seal is removable and re-sealable against a patient's skin.

In an embodiment, the seal is non-curing.

In an embodiment, the seal comprising butyl rubber, a filler, and a tackifying resin.

According to a third aspect, the invention described herein broadly consists in a kit of parts comprising the wound interface device according to the first aspect of the invention; and a liquid impermeable occlusive outer layer.

In an embodiment, the kit of parts further comprises an adhesive seal.

In an embodiment, a first removable release sheet is adhered to one side of the adhesive seal, and a second removable release sheet adhered to a second side of the adhesive seal, wherein the second removable release sheet is stretchable.

The kit of parts may include a connector for coupling to the inlet and outlet ports of the wound interface device.

In an embodiment, the connector is a two-part connector. The connector may include and inlet and outlet port interfacing portion, and a collar for holding the interfacing portion in place.

In an embodiment, the wound interface device comprises a boss having a chamfered surface, and wherein the connector comprises a complementary chamfered surface for sealing engagement with the chamfered surface of the boss.

In an embodiment, the connector and/or the device includes a locating feature for assisting with correctly orienting the connector relative to the device. The locating feature may include a retention feature for engaging with the connector. In some embodiments the locating feature includes a tab extending from a top surface of the device body.

According to a fourth aspect, the invention described herein broadly consists in a wound therapy system comprising a wound dressing according to the second aspect of the invention, and a pump coupled to the device outlet and configured to apply a negative pressure to the wound dressing.

The system may include a fluid input and a fluid output for connection to a wound treatment device located at the wound. The wound treatment device may be as described above. The fluid input is adapted to be fluidly connected to an upstream side of the wound treatment device and the fluid output is adapted to be fluidly connected to a downstream side of the wound treatment device.

In an embodiment, the occlusive layer is adhered to a patient's skin and over the wound therapy device.

In an embodiment, the wound therapy system further comprising a reservoir for collecting exudate removed from the dressing.

In an embodiment, the wound therapy system further comprising a reservoir for storing treatment fluids for administering to the wound.

In an embodiment, the pump is coupled to the wound dressing by way of a dual lumen conduit.

In an embodiment, the wound therapy system further comprises: an air inlet valve upstream of the wound treatment device inlet; an actuator to drive the air inlet valve between an open position and a closed position; a controller in communication with the actuator and a motor driving the pump to operate the air inlet valve and the pump; wherein the controller is configured to: i) open the air inlet valve and operate the pump to maintain a first vacuum pressure at the wound treatment device and introduce air into the wound treatment device; ii) close the air inlet valve and operate the pump to maintain a second vacuum pressure at the wound treatment device and remove air and fluid from the wound treatment device; and wherein the first vacuum pressure is less than or equal to the second vacuum pressure.

In an embodiment, the controller is configured to operate the pump to continuously maintain a negative pressure environment at the wound treatment device when the air valve is open and closed.

In an embodiment, the first and second vacuum pressures provide for effective negative pressure wound therapy.

In an embodiment, the controller is configured to repeat steps i) and ii) to cycle the air inlet valve between the open and closed positions.

In an embodiment, the controller is configured to operate the pump when the air inlet valve is open to maintain a substantially constant first vacuum pressure.

In an embodiment, the controller is configured to operate the pump when the air inlet valve is closed to maintain a substantially constant second vacuum pressure.

In an embodiment, the controller is configured to: in step (i), operate the pump with the air inlet valve open so that the system is in an equilibrium state with a zero or constant pressure differential across the treatment device; in step (ii), operate the pump with the air inlet valve closed so that the system is in an equilibrium state with a zero or constant pressure differential across the treatment device.

In an embodiment, the controller is configured to operate the air inlet valve between open and closed to introduce a flow rate of air into the system that generates a bubble flow or slug flow comprising bubbles or slugs of air entrained in fluid flow from the wound treatment device.

In an embodiment, the controller is configured to operate the air inlet valve between open and closed to reduce a density of fluid at the wound to lift the fluid from the wound against gravity.

In an embodiment, the controller is configured to open and close the air inlet valve periodically.

In an embodiment, in step i) the controller is configured to open the air inlet valve for a predetermined time period.

In an embodiment, in step i) the controller is configured to open the air inlet valve for at least 10 seconds.

In an embodiment, in step ii) the controller is configured to close the air inlet valve for a predetermined time period. In an embodiment, in step i) the controller is configured to open the air inlet valve for at least 10 seconds.

In an embodiment, the air inlet valve is open for at least 10% of the cycle pitch, or at least 20% of the cycle pitch, or at least 30% of the cycle pitch, or at least 40% of the cycle pitch, or at least 50% of the cycle pitch.

In an embodiment, in step i), the air inlet valve is open for a sufficient time period so that a volume of air delivered through the system is at least a substantial portion of a total volume of the system.

In an embodiment, in step (i), the air inlet valve is open for a sufficient time period so that the volume of air delivered to the system is at least 50%, or at least 100% of the total volume of the system.

In an embodiment, the first vacuum pressure is about 30% to 100% of the second vacuum pressure.

In an embodiment, the first vacuum pressure is about 30 to 100 mmHg, preferably about 30 to 70 mmHg.

In an embodiment, wherein the second vacuum pressure is about 100 to 150 mmHg.

In an embodiment, the first vacuum pressure is about 10 to 125 mmHg less than the second pressure, for example about 10 to 125 mmHg.

In an embodiment, in step (i) the controller is configured to operate the pump to achieve a vacuum pressure threshold.

In an embodiment, in step (ii) the controller is configured to operate the pump to achieve a vacuum pressure threshold.

In an embodiment, the system comprises: a downstream pressure sensor located downstream of the wound treatment device and in communication with the controller, and the controller is configured to, in step i) operate the pump to achieve the vacuum pressure threshold based on a pressure sensed by the downstream pressure sensor.

In an embodiment, the system comprises: an upstream pressure sensor located upstream of the wound treatment device and in communication with the controller, and the controller is configured to, in step ii), operate the pump to achieve the vacuum pressure threshold based on a pressure sensed by the upstream pressure sensor.

In an embodiment, the system comprises: an upstream pressure sensor located upstream of the wound treatment device and in communication with the controller, a downstream pressure sensor located downstream of the wound treatment device and in communication with the controller, and the controller is configured to, in step i) operate the pump to achieve a first vacuum pressure threshold based on a pressure sensed by the downstream pressure sensor; and in step ii), operate the pump to achieve a second vacuum pressure threshold based on a pressure sensed by the upstream pressure sensor.

In an embodiment, the first vacuum pressure threshold is less than or equal to the second vacuum pressure threshold.

In an embodiment, the system comprises an inlet restriction, and the upstream pressure sensor is located upstream of the inlet restriction so that the upstream pressure sensor measures ambient pressure when the air inlet valve is open.

In an embodiment, the system comprises a reservoir for collecting fluid removed from the wound, and wherein the reservoir is located downstream of the pump such that fluid removed from the wound passes through the pump to the reservoir.

In an embodiment, the reservoir comprises a flexible bag.

In an embodiment, the reservoir comprises a vent to vent the reservoir to the ambient atmosphere.

In an embodiment, the system includes an occlusive cover layer to cover the wound;
a fluid supply conduit in fluid communication with the fluid outlet, the fluid supply conduit having one or more supply conduit outlets;
a fluid removal conduit in fluid communication with the fluid inlet, the fluid removal conduit having one or more removal conduit inlets;

In an embodiment, the system comprises a treatment fluid inlet upstream of the fluid outlet to connect a supply of treatment fluid.

In an embodiment, the system is configured so that, in step i) the introduction of treatment fluid to the wound treatment device is prevented or reduced by the introduction of air to the wound treatment device by the first vacuum pressure, and in step ii), treatment fluid is drawn to the wound treatment device by the second vacuum pressure.

In an embodiment, the system comprises:
a treatment fluid valve between the treatment fluid inlet and the fluid outlet, and
an actuator to drive the treatment fluid inlet valve between an open position and a closed position, wherein the controller is in communication with the fluid inlet valve actuator and the controller is configured to, in a fluid supply state:
   iii). open the fluid inlet valve and operate the pump to maintain a vacuum pressure at the wound treatment device and introduce treatment fluid into the wound treatment device;
   iv). close the fluid inlet valve and operate the pump to maintain a vacuum pressure at the wound treatment device and remove fluid from the wound treatment device.

In an embodiment, the controller is configured to operate the pump to continuously maintain a negative pressure environment at the wound treatment device when the fluid inlet valve is open and closed.

In an embodiment, the controller is configured to, in step (iii), operate the pump to generate a third vacuum pressure at the wound treatment device, and, in step (iv), operate the pump to generate a fourth vacuum pressure at the wound treatment device, wherein the third vacuum pressure is less than or equal to the fourth vacuum pressure.

In an embodiment, the third vacuum pressure is equal or similar to the first vacuum pressure and the fourth vacuum pressure is equal or similar to the second vacuum pressure.

In an embodiment, the third and fourth vacuum pressures provide for effective negative pressure wound therapy.

In an embodiment, after closing the fluid inlet valve and operating the pump to generate the vacuum pressure at the wound, the controller is configured to:
(v) flush the treatment fluid from the wound by:
(v)(a) opening the air inlet valve and operating the pump to maintain a vacuum pressure (e.g. the first vacuum pressure) at the wound treatment device and introduce air into the wound treatment device, and
(v)(b) closing the air inlet valve and operating the pump to maintain a vacuum pressure (e.g. the second vacuum pressure) at the wound treatment device and remove fluid from the wound treatment device.

In an embodiment, in step (v) the controller is configured to repeat steps (v)(a) and (v)(b) a predetermined number of times (for example, three times) to remove treatment fluid from the wound.

In an embodiment, in the fluid treatment state, the controller is configured to repeat steps (iii) to (v) a predetermined number of times.

In an embodiment, the controller is configured to, in step (iv), close the fluid inlet valve, wait for a predetermined time period, and operate the pump to generate the vacuum pressure at the wound treatment device and remove fluid from the wound treatment device.

In an embodiment, the controller is configured to activate the fluid supply state periodically.

In an embodiment, a time period between activating the fluid supply state is much greater than a cycle time of the air inlet valve.

In an embodiment, the system comprises an upstream pressure sensor and/or a downstream pressure sensor in communication with the controller, and, in step (iii), the controller is configured to operate the pump to achieve a vacuum pressure threshold based on a pressure sensed by the upstream and/or downstream pressure sensor.

In an embodiment, the system comprises an upstream pressure sensor and/or a downstream pressure sensor in communication with the controller, and, in step (iv), the controller is configured to operate the pump to achieve a vacuum pressure threshold based on a pressure sensed by the upstream and/or downstream pressure sensor.

This invention may also be said broadly to consist in the parts, elements and features referred to or indicated in the specification of the application, individually or collectively, and any or all combinations of any two or more said parts, elements or features. Where specific integers are mentioned herein which have known equivalents in the art to which this invention relates, such known equivalents are deemed to be incorporated herein as if individually described.

The term ‘comprising’ as used in this specification and claims means ‘consisting at least in part of’. When interpreting statements in this specification and claims that include the term ‘comprising’, other features besides those prefaced by this term can also be present. Related terms such as ‘comprise’ and ‘comprised’ are to be interpreted in a similar manner.

It is intended that reference to a range of numbers disclosed herein (for example, 1 to 10) also incorporates reference to all rational numbers within that range and any range of rational numbers within that range (for example, 1 to 6, 1.5 to 5.5 and 3.1 to 10). Therefore, all sub-ranges of all ranges expressly disclosed herein are hereby expressly disclosed.

As used herein the term ‘(s)’ following a noun means the plural and/or singular form of that noun. As used herein the term ‘and/or’ means ‘and’ or ‘or’, or where the context allows, both.

### BRIEF DESCRIPTION OF THE DRAWINGS

The present invention will now be described by way of example only and with reference to the accompanying drawings, in which:

FIG. 1 is a schematic view of a prior art NPWT system;

FIG. 2 is a cut-away side view of a prior art device at a wound site, illustrating the provision of fluid flow to the device;

FIG. 3 is a is a cut-away side view of a prior art device of FIG. 2, illustrating the removal of fluid flow from the device;

FIG. 4 is a schematic view of a NPWT system;

FIG. 5 is a top perspective view of one embodiment wound treatment device;

FIG. 6 is an underside perspective view of the device of FIG. 5;

FIG. 7 is an underside perspective view of the device of FIGS. 5 and 6, illustrating example fluid flow through the device;

FIG. 8 is a top perspective view of FIG. 5 illustrating a cut away along lines YY;

FIG. 9 is a side angular view of the device of FIG. 5 through lines YY;

FIG. 10 is a top elevation view of the device of FIG. 5, showing how the device may be trimmed to fit a particular treatment site;

FIG. 11 is a top elevation view of the device trimmed as illustrated in FIG. 10;

FIG. 12 is a top perspective view of the trimmed device of FIG. 10;

FIG. 13 is an underside perspective view of the trimmed device of FIGS. 10;

FIG. 14 is a side section view of the device, taken along an axis of the device inlet channel, illustrating fluid flow through the device and wound site;

FIG. 15 is a side perspective view of the trimmed device of FIGS. 10 to 13, taken along an axis of the device inlet channel, illustrating fluid flow through the device and wound site;

FIG. 16 is a cut-away side view illustrating the placement of the device of FIGS. 5 to 15 at a wound site;

FIG. 17 is a cut-away side view corresponding to FIG. 16, illustrating the step of placing an occlusive layer over the device and wound site;

FIG. 18 is a cut-away side view corresponding to FIGS. 16 and 17, illustrating the occlusive layer in place over the device and wound site;

FIG. 19 is a cut-away side view illustrating a wound site having a bioresorbable material placed within it;

FIG. 20 is a cut-away side view corresponding to FIG. 19, illustrating the step of placing an occlusive layer over the device and wound site with bioresorbable material;

FIG. 21 is a cut-away side view corresponding to FIGS. 19 and 20, showing the device of FIGS. 5 to 15 positioned over the bioresorbable material and illustrating the step of placing the occlusive layer over the device;

FIG. 22 is a schematic section view illustrating a second embodiment NPWT device installed at a wound site;

FIG. 23 is a schematic section view illustrating a third embodiment NPWT device installed at a wound site;

FIG. 24 is a schematic section view illustrating a fourth embodiment NPWT device installed at a wound site,

FIG. 25 is a side section view of a portion of the device of FIGS. 5 to 15, taken along a cross sectional axis of the device, illustrating the cross-sectional profile of the spacers;

FIG. 26 is a top perspective view of a further (fifth) embodiment wound treatment device;

FIG. 27 is an underside perspective view of the device of FIG. 26;

FIG. 28 is a detail view of the connection between the device inlet and outlet ports to the body of the device of FIGS. 26 and 27;

FIG. 29 is an exploded detail view corresponding to FIG. 28;

FIG. 30 in an underside view of the device of FIG. 26 to 29, illustrating exemplary fluid flow paths for flow of treatment fluid along the underside of the device;

FIG. 31 is a top plan view of the device of FIGS. 26 to 30;

FIG. 32 is section view taken through line AA of FIG. 31, showing flow through the device inlet;

FIG. 33 is section view taken through line BB of FIG. 31, showing flow through the device outlet;

FIG. 34 is a schematic side section view showing an alternative shape for the cross-sectional profile of spacers, and illustrating the changing profile of the spacers under a compressive load;

FIG. 35 is a cut-away end view of the device of FIGS. 26 to 29, with the inlet and outlet connector removed, showing the section profile of the inlet channel;

FIG. 36 is a cut-away side elevation view of the device of FIGS. 26 to 29, showing the section profile of the outlet channel;

FIG. 37 is a top detail view of the connection between the device inlet and outlet ports to the body of an alternative embodiment device;

FIG. 38 is an underside view corresponding to FIG. 37

FIG. 39 is a top perspective view of the connection of the inlet and outlet ports to the body of the device of the embodiment of FIGS. 37 and 38;

FIG. 40 is an exploded perspective view of the connection shown in FIG. 39;

FIG. 41 is an underside perspective view of the connector component of the embodiment shown in FIGS. 37 to 40;

FIG. 42 is a top perspective view of the connector component of the embodiment shown in FIGS. 37 to 41;

FIG. 43 is section view taken through the device of FIGS. 37 to 42, through the device inlet, showing flow through the inlet;

FIG. 44 is section view taken through the device of FIGS. 37 to 42, through the device outlet, showing flow through the outlet;

FIG. 45 is a top detail view of the connection between the device inlet and outlet ports to the body of a further alternative embodiment device;

FIG. 46 is an underside view corresponding to FIG. 45;

FIG. 47 is a top perspective view of the connection of the inlet and outlet ports to the body of the device of the embodiment of FIGS. 45 and 46;

FIG. 48 is an exploded perspective view of the connection shown in FIG. 47;

FIG. 49 is an underside perspective view of the connector component of the embodiment shown in FIGS. 45 to 48;

FIG. 50 is a top perspective view of the connector component of the embodiment shown in FIGS. 45 to 48;

FIG. 51 is section view taken through the device of FIGS. 45 to 48, through the device inlet, showing flow through the inlet;

FIG. 52 is section view taken through the device of FIGS. 45 to 48, through the device outlet, showing flow through the outlet;

FIG. 53 is a top detail perspective view of the connection between the device inlet and outlet ports to the body of a further (eighth) alternative embodiment device;

FIG. 54 is an top view corresponding to FIG. 53;

FIG. 55 is a section view taken along line F55 of FIG. 54;

FIG. 56 is a top-end perspective view corresponding to FIG. 53;

FIG. 57 is an exploded perspective view of the connection shown in FIG. 53;

FIG. 58 is a top perspective view of the connector component of the embodiment shown in FIGS. 53 to 57;

FIG. 59 is an underside perspective view of the connector component of the embodiment shown in FIGS. 53 to 57;

FIG. 60 is section view taken through the device of FIGS. 53 to 57, through the device inlet, showing flow through the inlet;

FIG. 61 is section view taken through the device of FIGS. 53 to 57, through the device outlet, showing flow through the outlet;

FIG. 62 is a top perspective view of a ninth embodiment device;

FIG. 63 is an underside perspective view of the device of FIG. 62;

FIG. 64 in an underside view of the device of FIGS. 62 and 63, illustrating exemplary fluid flow paths for flow of treatment fluid along the underside of the device;

FIG. 65 is a right side elevation view of the device of FIGS. 62 to 64;

FIG. 66 is a left side elevation view of the device of FIGS. 62 to 64;

FIG. 67 is a schematic side section view showing the cross-sectional profile of spacers in the device of FIGS. 62 to 66, and illustrating the changing profile of the spacers under a compressive load;

FIG. 68 is a top perspective view of a ninth embodiment device having a split tube configuration;

FIG. 69 in an underside perspective view of the device of FIG. 68;

FIG. 70 is an underside view of the device of FIGS. 68 and 69;

FIG. 71 is a cut away end view of the device of FIGS. 68 to 70, showing the inlet channel;

FIG. 72 is a cut away side elevation view of the device of FIGS. 68 to 70;

FIG. 73 is a schematic side section view showing the cross-sectional profile of spacers in the device of FIGS. 68 to 70, and illustrating the changing profile of the spacers under a compressive load;

FIG. 74 is a top perspective view of a tenth embodiment device having an alternative embodiment connector portion at the inlet and outlet ports, with the connector omitted;

FIG. 75 is an end elevation view of the connection boss of the embodiment shown in FIG. 74, with the connector omitted;

FIG. 76 is a side cut-away view of the connection boss of FIG. 75;

FIG. 77 is an end cut-away view of the connection boss of FIGS. 75 and 76 assembled with the connector;

FIG. 78 is a side elevation view of the connection boss of FIGS. 75 to 77 assembled with the connector;

FIG. 79 is a view corresponding to FIG. 76, but with inlet and outlet conduits coupled to the connector;

FIG. 80 is an exploded view corresponding to FIG. 79;

FIG. 81 is a detail, top view of the connector portion of the device of FIG. 74

FIG. 82 is a top perspective view of a further (eleventh) embodiment device having an alternative embodiment connector portion at the inlet and outlet ports;

FIG. 83 is an end elevation view of the inlet and outlet connector portion of the embodiment shown in FIG. 82;

FIG. 84 is a side cut-away view of the connector portion of FIG. 83;

FIG. 85 is an underside perspective view of a twelfth embodiment device having alternatively shaped spacers;

FIG. 86 is a top perspective view of the device of FIG. 85;

FIG. 87 is a side elevation view of the device of FIGS. 85 and 86, with a cut-away portion;

FIG. 88 is a side elevation detail view of one of the spacers from the device of FIGS. 85-87, with part of the spacer cut away;

FIG. 89 is an underside perspective view of a portion of a device having a further embodiment spacer;

FIG. 90 is a top perspective view corresponding to FIG. 89;

FIG. 91 is a side elevation detail view of one of the spacers from the device of FIGS. 89 and 90, with part of the spacer cut away;

FIG. 92 is an underside perspective view of a portion of a device having yet a further alternative embodiment spacer;

FIG. 93 is a top perspective view corresponding to FIG. 92;

FIG. 94 is an underside view corresponding to FIG. 92;

FIG. 95 is a side section detail view through two of the spacers from the device of FIGS. 92 to 94;

FIG. 96 is an underside perspective view of a portion of a device having yet a further embodiment spacer;

FIG. 97 is a top perspective view corresponding to FIG. 96;

FIG. 98 is an underside view corresponding to FIG. 96;

FIG. 99 is a side section detail view through four of the spacers from the device of FIGS. 96 to 98;

FIG. 100 is a cut-away side view illustrating a wound site with undermining having a multiplicity of small filler pieces of bioresorbable material placed within it to fill the cavities created by the undermining.

FIG. 101 is a cut-away side view corresponding to FIG. 100, illustrating the step of placing a sheet of bioresorbable material over the small filler pieces and wound site;

FIG. 102 is a cut-away side view corresponding to FIGS. 100 and 101, showing the device of FIGS. 5 to 15 positioned in the wound site, and illustrating the step of placing an occlusive layer over the device;

FIG. 103 is a cut-away side view corresponding to FIGS. 100 to 102, illustrating the occlusive layer in place over the device and wound site;

FIG. 104 provides a high-level schematic representation of a negative pressure treatment (NPT) system according to at least one embodiment described herein;

FIG. 105 illustrates the system of FIG. 104 applied to an external wound;

FIG. 106 is a schematic representation of a vacuum unit of the system of FIG. 104.

FIG. 107 is a schematic representation of the system of FIG. 104;

FIG. 108 is a schematic representation of the system of the negative pressure treatment (NPT) system shown in FIG. 4; and

FIG. 109 is a schematic representation of a further alternative configuration for the negative pressure treatment (NPT) system of FIG. 4.

### DETAILED DESCRIPTION

### I. Definitions

The term “extracellular matrix” (ECM) as used herein refers to animal or human tissue that has been decellularized and provides a matrix for structural integrity and a framework for carrying other materials.

The term “decellularized” as used herein refers to the removal of cells and their related debris from a portion of a tissue or organ, for example, from ECM.

The term “polymeric material” as used herein refers to large molecules or macromolecules comprising many repeated subunits, and may be natural materials including, but not limited to, polypeptides and proteins (e.g. collagen), polysaccharides (e.g. alginate) and other biopolymers such as glycoproteins, or may be synthetic materials including, but not limited to, polypropylene, polytetrafluoroethylene, polyglycolic acid, polylactic acid, and polyester.

In this specification and claims, the terms ‘negative pressure’ and ‘vacuum pressure’ may be used interchangeable to mean a gauge pressure less than an ambient pressure and an absolute pressure less than atmospheric pressure. Alternative terms include ‘sub-atmospheric pressure’, ‘suction pressure’ or ‘reduced pressure’. For example, a negative pressure or vacuum pressure of 100 mmHg is −100 mmHg gauge pressure or around 660 mmHg absolute pressure. The terms ‘higher’, ‘increase’, when used in relation to negative or vacuum pressure, are intended to mean higher or increasing negative pressure. For example, a gauge pressure of −150 mmHg (610 mmHg absolute) is higher than a gauge pressure of −100 mmHg (660 mmHg absolute). Similarly, in relation to the terms ‘lower’, ‘decrease’, when used in relation to negative or vacuum pressure, are intended to mean lower or decreasing negative pressure. For example, a gauge pressure of −100 mmHg is lower than a gauge pressure of −150 mmHg.

In this specification and claims, unless the context indicates otherwise, the term ‘exudate’ is intended to mean any fluid removed from a wound site of a patient. For example, exudate may comprise fluid produced by the patient, and/or fluid applied to the wound site by the system, including air or treatment fluid such as saline, or fluid providing medication, or fluid from a surgical intervention that may have introduced or administered treatment fluids to the wound site via a separate route such as by injection.

### II. Device

Various embodiments will now be described with reference to FIGS. 4 to 99. In these figures, unless otherwise described, like reference numbers are used in different embodiments to indicate like features, with the addition of a multiple of 100. Directional terminology such as the terms ‘front’, ‘rear’, ‘upper’, ‘lower’, and other related terms are used in the following description for ease of description and reference only, it is not intended to be limiting.

The invention generally provides a wound interface device 1 for use in negative pressure wound therapy. The device 1 has a flexible body 3 with a plurality of spacers 5 for creating a therapy space between a wound surface and a wound facing surface 7 of the device body 3. The therapy space 43 facilitates the delivery and removal of fluids from the wound, and the application of negative pressure to the wound. The fluids may be a gas or liquid or both. The wound interface device 1 forms part of a multi-layer wound dressing 27 (FIG. 18) that includes a liquid impermeable occlusive layer 45 for adhering around the peri wound 49 to provide an enclosed environment around the wound.

The device 1 comprises an inlet 11 for the instillation of fluids such as treatment fluids to the wound site. The inlet 11 comprises a passage configured to deliver fluid from an inlet port 13 to an area adjacent a first end 2a of the device 1. In the embodiment shown, the inlet 11 comprises a channel provided in the body of the device 1 that is closed on the wound facing surface 7 of the channel and open to a top surface 9 of the device. The inlet channel 11 extends in a first, preferably longitudinal direction of the device, terminating at an inlet opening 11a at or near the first end 2a of the device 1. Alternatively, the inlet 11 may comprise a tubular conduit that is integral or attached to the device body, as will be described in more detail below in relation to further exemplary embodiments.

An outlet 15 is provided for the removal of fluids from the wound therapy space 43. The outlet 15 is configured to extract fluid from adjacent a second end 2b of the device 1, opposite the first end 2a. In the embodiment shown, the outlet 15 comprises a channel, provided in the body of the device with a relatively narrow outlet channel opening 16 open to the underside of the device, and closed along a top surface 9 of the device. The outlet 15 including outlet channel opening 16, extends in a second, preferably transverse direction of the device (perpendicular to the inlet channel 11), and opens towards the first end 2a of the device. The outlet channel including outlet channel opening 16 preferably extends across a major part of the width of the device 1, to draw fluid from across the full width of the device into the outlet 15. The outlet channel opening 16 preferably is provided in substantially close proximity to the wound surface 42. Being provided in close proximity to the wound surface 42 allows the outlet 15 to extract a significant portion of the treatment fluid and exudate from the therapy space 43, reducing the pooling of fluids within the therapy space 43.

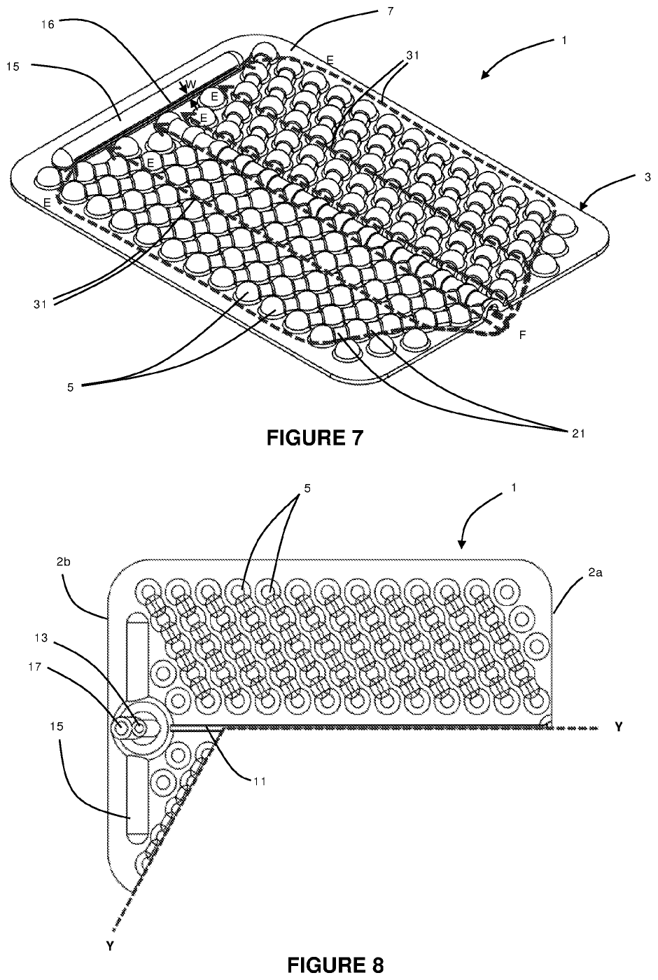

The outlet channel opening 16 is an elongate slot-like opening with and opening width W (FIG. 7) that is substantially less than the radius of the outlet channel 15. Preferably the width W of the outlet channel opening is less than about half of the radius

In the embodiments shown, the outlet channel opening 16 is a substantially linear slot, extending along a majority of the width of the device 1. In alternative embodiments, the outlet channel opening 16 may have another shape, for example the outlet channel opening 16 may be undulating, for example it may be shaped as a repeating a sinusoidal wave where the opening 16 alternates between a first position and a second position, where the first position is substantially proximate the wound surface and the second position is substantially proximate the wound facing surface of the device. Such an embodiment may provide additional benefit in allowing fluids to be collected from wound surface while ensuring a fluid flow path is maintained in the event that tissue debris and wound slough obstruct the outlet channel opening.

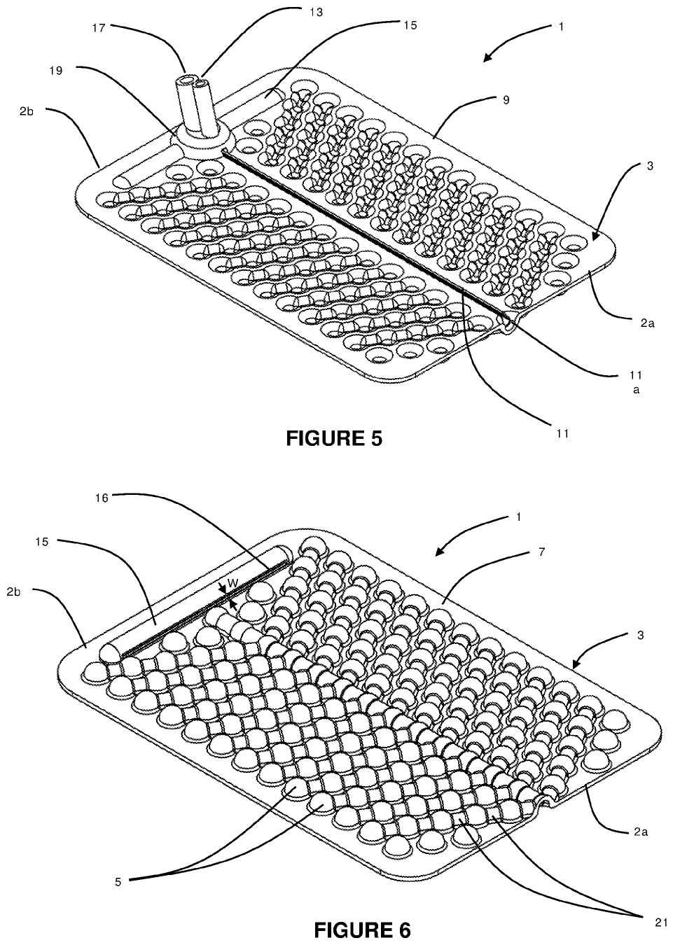

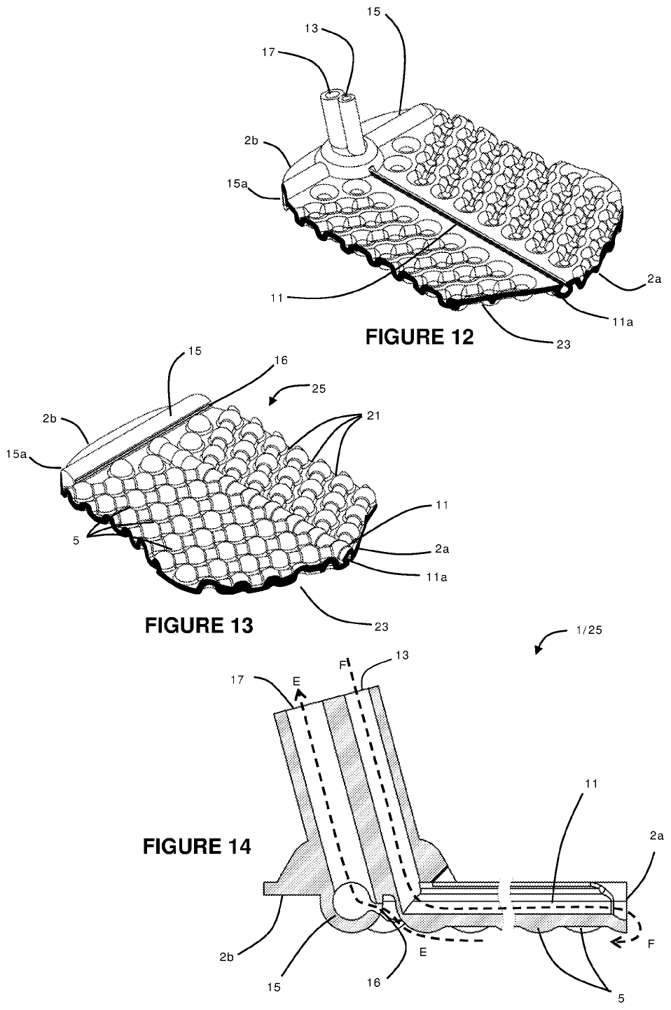

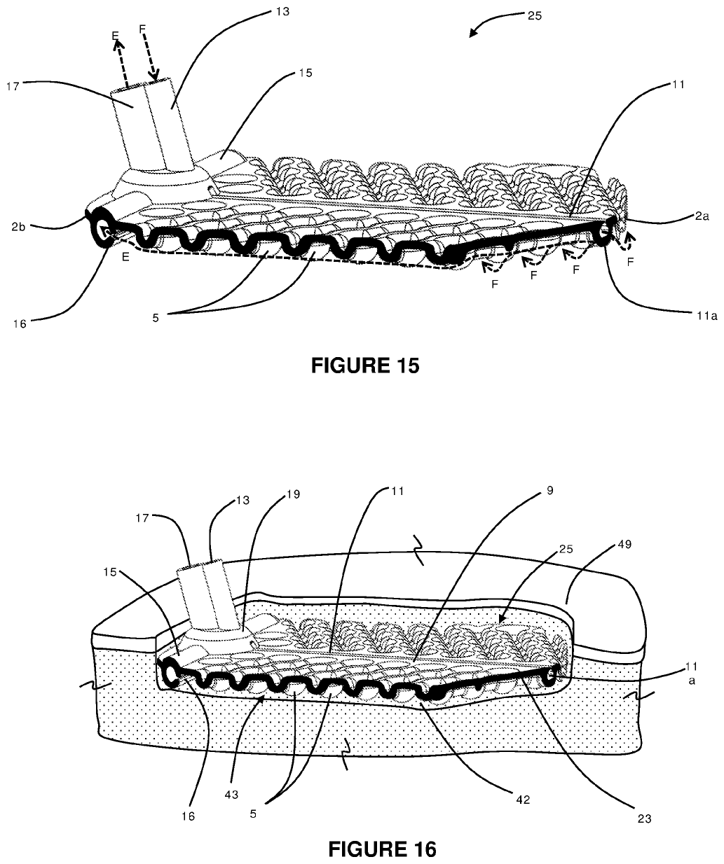

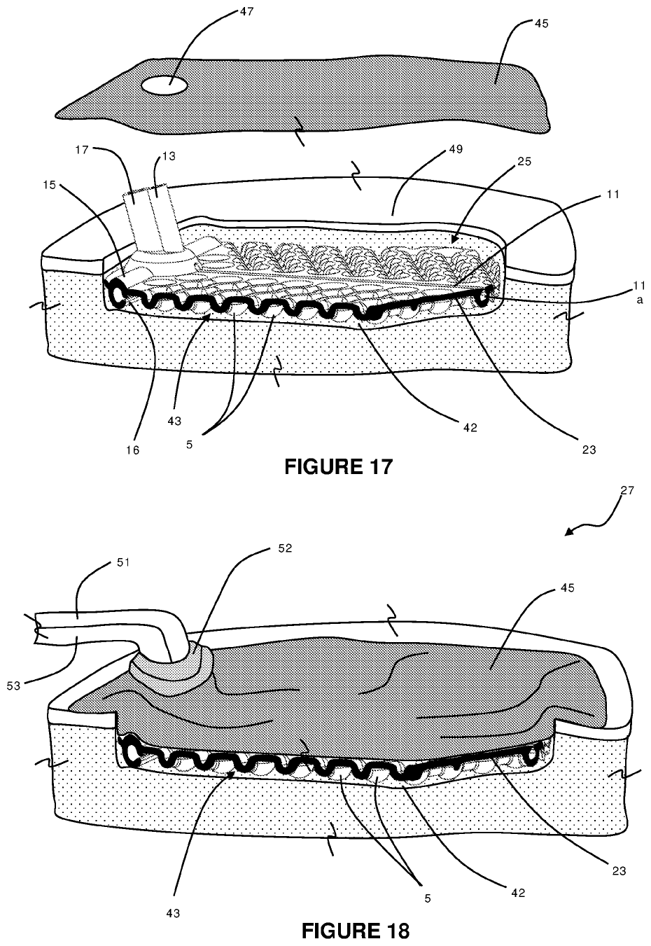

In the embodiments shown herein, the width W of the outlet channel opening 16 is substantially uniform along the length of the outlet channel 15, however it will be appreciated that in alternative embodiments, the width W of the outlet channel opening 16 may vary along the width of the device. Preferably, the width W of the outlet channel opening is between about 0.2 mm to about 3 mm. More preferably, the width W of the outlet channel opening is between about 0.5 mm to about 1 mm. In the embodiment shown in FIGS. 5 to 21, the width W of the outlet channel opening is about 0.6 mm wide at the narrowest point. As will be described further below, the device is typically used in a system having a negative pressure pump and reservoir fluidly connected to and downstream of the device outlet port 17. The width W of the outlet channel opening 16 will typically be the smallest cross-sectional dimension along the fluid pathway from the device downstream to the pump or reservoir. This minimises the likelihood of blockages occurring downstream of the outlet channel opening 16.

The narrow, elongate nature of the outlet channel opening 16 provides a higher likelihood that the outlet 15 will be in fluid communication with fluid that may be pooling at the wound surface 42 compared to an outlet provided at a single point. The narrow opening acts as a constriction to reduce the likelihood of blockages by preventing the passage of large blood clots, fibrin and other solidified fluids or tissue debris of a size that may cause a blockage of the outlet conduit 53, or the outlet channel 15. Some blood, fibrin and other particles may still pass through the outlet channel opening 16, depending on the width W of the outlet channel opening. However, in preferred embodiments, any large blood clots, fibrin and other solidified fluids or tissue debris that would potentially terminally block the tubing are not able to pass through the outlet channel opening 16 into the outlet 15. Further, if such large blood clots, fibrin and other solidified fluids or tissue debris are drawn against the outlet channel opening 16 under vacuum pressure, because the outlet channel opening runs along the width of the device, a block in a portion of the outlet channel opening 16 is unlikely to adversely impact on the application of negative pressure to the wound site, as this is distributed across the width of the device and throughout the connected treatment space 43.

The outlet 15 is configured to deliver fluid to an outlet port 17 of the device, which is for coupling to a negative pressure source.

The inlet port 13 is configured for coupling to a fluid source and the outlet port 17 is configured for coupling to a negative pressure source. The fluid source and/or the negative pressure source may be provided to the device via an external conduit. In the embodiment shown, the inlet and outlet ports 13, 17 are provided extending from a moulded member 19. The moulded member 19 projects from an opposite side of the device body 1 to the wound facing side 7, that is from the top surface 9 of the device 3, for extending through the occlusive layer 45 of the dressing and to facilitate coupling to the external fluid source and/or negative pressure source. A tapered and raised surface on the moulded member 19 is provided to facilitate a push-fit connection with the external conduit(s), however other connections are envisaged. Having projecting inlet and outlet ports 13, 17 that are integral with the device body allows the connection of the device to supply and removal conduits to be made away from the wound. This is advantageous as rigid components positioned close to the wound can cause pain and discomfort by pressing on the wound, and in some cases may cause pressure sores where pressure of the rigid component is maintained.

Referring to FIG. 7, the spacers 5 define multiplicity of fluid pathways 31 between the first end 2a of the device 1 and the second end 2b of the device. The fluid pathways 31 are formed between the wound facing surface 7 of the device and the wound surface 42, to allow treatment fluids from the inlet 11 and wound fluids such as wound exudate to flow towards and into the outlet 15, before exiting the outlet port 17. Having a multiplicity of pathways 31 ensures that the device 1 is able to continue to draw fluid from the wound even if one pathway becomes blocked.

The spacers 5 are shaped such that only a portion the spacer is configured contact the wound surface 42. In the embodiment shown, the spacers 5 are bulbous projections having a convex wound contacting surface, however, other shaped spacers are envisaged and some are described below. The spacers 5 are shaped and distributed to allow the device 1 to flex and therefore conform to the wound site to ensure healthy granulation tissue forms at the contact point between the spacer 5 and the wound surface 42. The spacers 5 also preferably ensure a therapy space 43 is substantially maintained between the wound surface and the device 1 under various loading conditions, for example in circumstances when the device 1 is subjected to a range of negative pressures, or when a patient moves or distributes their weight so as to put physical pressure on the device 1. The spacers 5 are shaped and distributed to prevent the substantial collapse of the spacers 5, for example through the buckling of the spacer walls, to thereby maintain the therapy space 43 under the application of negative pressure. Therefore, the spacers 5 must be sufficiently large, of sufficient wall thickness and curvature, and in sufficient number to prevent substantial collapse of the spacers 5 and to thereby maintain the therapy space 43 under the application of negative pressure. However, the spacers 5 must not be so large or densely distributed as to prevent the device 1 flexing to comply with the contours of a wound surface 42 or as to prevent the passage of fluids in the therapy space 43 from the first end 2a to the second end 2b.

Pressures for Negative Pressure Wound Therapy provided by the device 1 are preferably between about 30 mmHg to about 200 mmHg, and more preferably about 125 mmHg. The pressure drop from the first end 2a to the second end 2b is minimal or negligible, this enables the device described herein to provide therapy at a lower operating negative pressure compared to the prior art.

In some embodiments which will be described in more detail below, the device 1 may be used in a wound treatment system in combination with a bioresorbable layer, the bioresorbable layer being placed between the device and the wound surface. The spacers 5 are sized and arranged to prevent the bioresorbable layer lifting from the wound surface under negative pressure. The spacers 5 are preferably distributed in sufficient density across the wound facing surface 7 of the device to prevent the bioresorbable layer lifting from the wound surface under negative pressure.

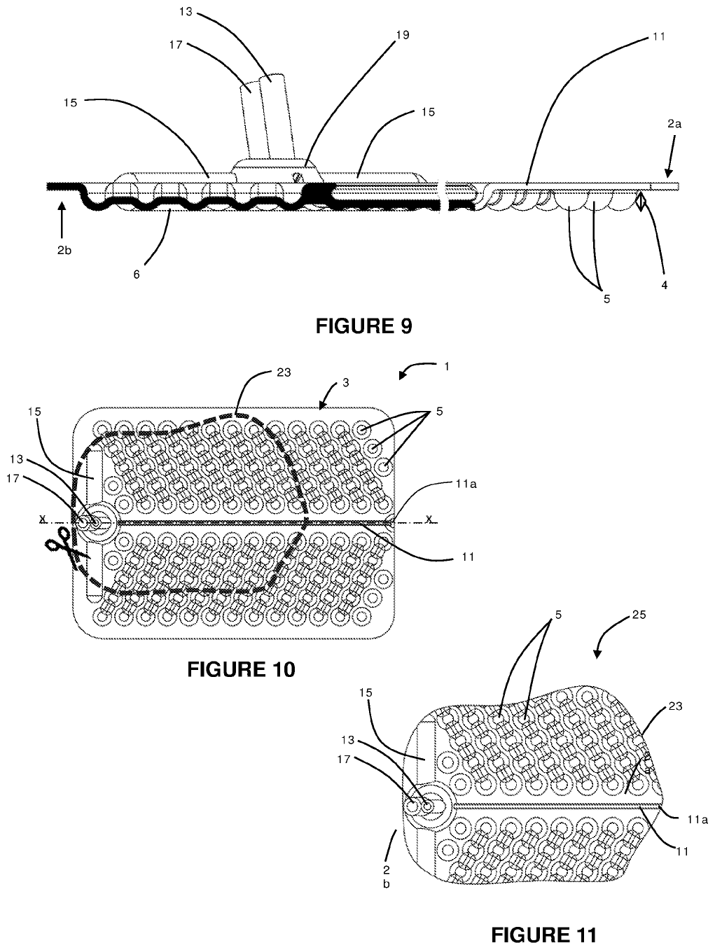

With reference to FIGS. 5 to 9, the spacers 5 extend from the wound facing surface 7 of the body 3 of the device 1, terminating at an apex of the spacer. The spacers have a spacer height 4 (FIG. 9) that is preferably between about 1 mm and about 10 mm, more preferably 2 mm to 5 mm, and in a preferred embodiment is about 3 mm.

The spacers 5 may be shaped, spaced, and/or secondary features may be present to assist with the distribution of treatment fluids and/or negative pressure across the wound site. In the device 1 shown in FIGS. 5-21, bridges 21 are provided between adjacent spacers 5 and configured to distribute treatment fluids. In this embodiment, the bridges 21 form a chevron-type pattern, to assist the direction of treatment fluids from the inlet opening 11a laterally across the wound surface and towards the second end 2b of the device, for example, across the width of the device and/or wound surface.

The secondary features may alternatively comprise ribs or differently shaped spacers, for example non-symmetric spacers 5.

The secondary features such as the bridges 21 preferably do not contact the wound surface, allowing the flow of fluids between the bridge surface and the wound surface. That is, the bridges 21 provide a gap 6 between the wound surface and the device to allow the passage of fluid and negative pressure.

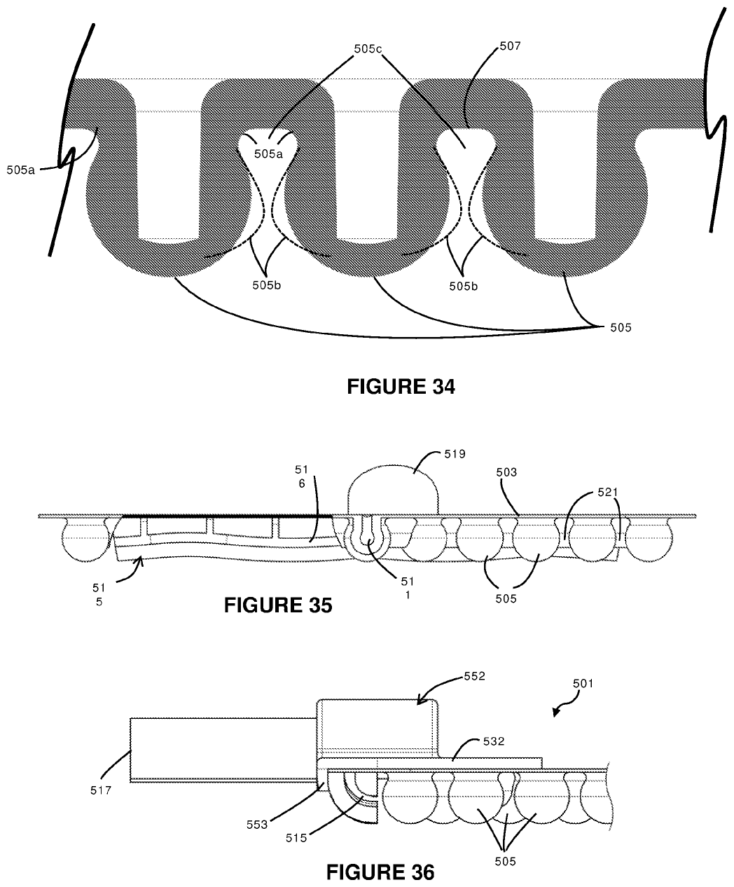

FIGS. 25 and 34 show side section views of a portion of the device, taken along a cross sectional axis of the device, showing the spacers 5 according to embodiments. The profile of the spacers 5 in the embodiment shown in FIG. 25 is more rounded, and sinusoidal, while the profile of the spacers 5 in the embodiment shown in FIG. 34 has more of a bulbous or undercut shape. The spacer height 4 is shown.

The height 4 of the spacer 5 and the gap 6 between the bridge 21 and the wound surface define the therapy space 43. For a portable and battery powered negative pressure treatment system it is most preferable to have the smallest therapy space 43 possible to ensure negative pressure treatment is administered across the wound while providing a sufficient means to allow the supply of fluids to, and removal of fluids from, the wound in order to promote wound healing for the required duration. If the height 4 of the spacer 5 and the gap 6 between the bridge 21 and the wound is too small, fluid and debris from the wound may block the device and prevent the administration of treatment fluid and negative pressure to the wound.

The gap 6 between the wound surface and the wound facing surface of the body 3 of the device 1 is determined by the spacer height 4 and is preferably between about 0.01 mm and about 10 mm, more preferably between about 0.1 mm and about 3 mm, and in a preferred embodiment is about 1 mm. The gap between the wound surface and the surface of the bridges 21 is less than the spacer height. When the total free volume of the therapy space 43 is enlarged, a larger amount of treatment fluid may be required to provide the same level of treatment as a device with less total free volume. An enlarged therapy space 43 will also require more energy to be provided to the vacuum pump assembly to pressurise the entire system to the desired level of vacuum pressure. This added demand of energy may shorten the period of time that a battery powered negative pressure treatment pump can operate.

In the device 1 shown in FIGS. 5-21, the spacers 5 are substantially round in transverse cross-section. In embodiments such as that shown having substantially round spacers 5, the spacers 5 have a diameter which is preferably between about 2 mm and about 10 mm, more preferably between about 3 mm and 7 mm, and in a preferred embodiment, is about 5 mm. In the embodiment shown, the spacers 5 are uniformly sized, however in alternative embodiments, the spacers 5 may have different sizes depending on their location in relation to the wound surface, for example, a spacer or spacers having a greater diameter and/or spacer height may be located at a first portion of the wound surface, and a spacer or spacers having a lesser diameter and/or spacer height may be located at a second portion of the wound surface.

The spacers 5 and/or the secondary features may further assist in the provision of therapy to or in the healing of the wound. For example, the spacers 5 and/or secondary features, and the wall thickness of the body of the device may be sufficiently flexible and shaped such that the rows of spacers contract towards the adjacent other at least partially when under negative pressure, such that the area of the device reduces. This contraction may pull the wound surface into an at least partially contracted state, in the width and/or lengthwise directions of the device. When negative pressure is removed or reduced, for example when the air inlet valve is opened and closed periodically for predetermined time periods, in a predetermined manner that generates a bubble flow or slug flow, the contracted state of the spacers 5 and/or secondary features, and thus the contracted state of the wound surface is released. The contraction of the wound site and subsequent release of the contraction is desirable to increase of blood flow to the wound surface, which aids in the healing process. However, it is also desirable for the fluid channels across the wound site to remain open even while the device and wound site are under contraction. In prior art devices, applied negative pressures often cause the device to contract in depth, obstructing fluid pathways through the device.

To illustrate this effect, experiments were carried out placing samples of known foam for NPWT in a polyurethane bag, applying a negative pressure, and observing the behaviour and contraction of the devices. The same experiment was carried out with a device similar to the one illustrated in FIG. 70, comprising rows of spacers arranged in a repeating chevron pattern with the chevron rows spaced 6 mm apart, angled at 60 degrees and with the apex of the chevron positioned along the central longitudinal axis of the device. The spacers were 5 mm in diameter and shaped with an undercut bulbous shape, similar to the shape illustrated in FIG. 73. The spacers were spaced 6 mm apart along the chevron rows and adjacent spacers in a row were connected via a bridge.

The device had an overall longitudinal width of about 110 mm, a traverse height of about 76 mm and an overall depth of about 9 mm. The spacers and bridge had a substantially constant wall thickness of about 1 mm, with cavities in the top surface of the device formed by the spacers as shown in FIGS. 5-21. The overall test device weighed 14.47 grams.

Tests were carried out for the device described above, a Granufoam™ reticulated polyurethane foam dressing, and a Whitefoam™ polyvinyl alcohol foam. The Granufoam™ dressing was tested in a dry state while the Whitefoam™ polyvinyl alcohol foam was hydrated prior to testing in accordance with the manufacturer's instructions for use.

Each test was conducted by placing the respective test device/dressing in a sealed polyurethane bag. A conduit passing through the bag was positioned centrally over the device and connected to an adjustable source of negative pressure. The length (longitudinal direction), width (transverse dimension), and depth of each test specimen were measured at system pressure levels of 0 mmHg (ambient), 50 mmHg and 150 mmHg. The results from this testing are shown in the table below.

| | | | | Contraction at | Contraction at |
|---|---|---|---|---|---|
| | | | | 50 mmHg from | 150 mmHg from |
| | 0 mmHg | | | original shape | original shape |
| | (atmospheric) | −50 mmHg | −150 mmHg | (%) | (%) |
| Granufoam ™ (PU) | | | | | |
| length (mm) | 100 | 82 | 82 | 18% | 18% |
| width (mm) | 104 | 96 | 96 | 8% | 8% |
| depth (mm) | 25 | 13 | 10 | 48% | 60% |
| Whitefoam ™ (PVA) | | | | | |
| length (mm) | 140 | 139 | 139 | 1% | 1% |
| width (mm) | 96 | 96 | 95 | 0% | 1% |
| depth (mm) | 8.75 | 6.6 | 5 | 25% | 43% |
| Present device | | | | | |
| length (mm) | 110 | 106 | 104 | 4% | 5% |
| width (mm) | 76.3 | 75.7 | 74.6 | 1% | 2% |
| depth (mm) | 4.6 | 4.6 | 4.6 | 0% | 0% |

The device body 3 is liquid and air impermeable. That is, the device 1 does not contain apertures or pores to allow the transmission of liquid or gas from the wound contacting side of the device to the top of the device 1, other than via the outlet 15. This facilitates the formation of the negative pressure therapy space 43, and ensures that the fluid flowing along the inlet channel 11, via the inlet port 13, is maintained on the top surface of the device 9 until it reaches the inlet opening 11a at the end of the device.

The device body 3, including the spacers 5, comprises a flexible, preferably resilient material, such as an elastomer. This enables the device to flex to conform to the contours of various wound surfaces. Preferably the device comprises silicone, and may comprise a low adhesion silicone coating to further ensure the wound does not adhere to device 1. Alternatively, the device 1 may comprise any other suitable elastomeric materials as will be apparent to one skilled in the art, for example, it may comprise one or more of polyurethane, thermoplastic polyurethane, thermoplastic elastomers, thermoplastic vulcanizates and/or rubber blends.

The device body 3 has a wall thickness that is selected to provide the required flexibility, resistance to compressive loads, and durability. The wall thickness of the device body in the embodiment shown is preferably between about 0.01 mm and about 10 mm, more preferably between about 0.1 mm and 5 mm, and in a preferred embodiment, is about 1 mm. In the embodiment shown, the device body 3 has a uniform wall thickness throughout. In alternative embodiments, wall thickness may vary across different portions of the device body. The wall thickness of the spacers 5 may be the same as or different to the thickness of the device body and/or the same as or different to the wall thickness of the bridges 21.

In the embodiment shown, the spacers 5 are positioned such that they are spaced apart from each other in a repeating pattern. The spacing between spacers 5 is preferably between about 2 mm and about 20 mm, more preferably between about 5 mm and 10 mm, and in a preferred embodiment, is about 7 mm. In the embodiment shown, the spacers 5 are located in rows, each row running along a first axis that is positioned approximately 60 degrees relative to the longitudinal axis of the inlet 11, and extending outward from the inlet 11 towards the edge of the device body 3, however other arrangements are anticipated.

Referring to FIGS. 10 to 13, the device 1 is trimmable to fit within the perimeter of various wound sites. As illustrated in FIG. 10, the device 1 may be trimmed to a size to match a given wound. When trimming to size, the trim line 23 may extend across the inlet channel 11 to shorten the device, and/or across the outlet channel 15 to narrow the device, if necessary. It is important that the trimmed device 25 (FIG. 11) encompasses the inlet and outlet ports 13, 17, as illustrated.

FIGS. 11 to 13 show the trimmed device 25. In this device, the inlet channel 11 terminates at the first end 2a of the device, such that any treatment fluid delivered to the wound will enter the therapy space between the first end 2a of the device and the edge of the wound. The first end 2a of the device may be defined by where the device terminates at the trim line 23. The outlet 15 is configured to extract fluid from adjacent a second end 2b of the device, which is opposite the first end 2a. In the example shown, at one point the cut line extends through the outlet channel 15 causing the outlet channel 15, to open to and terminate at a side edge 15a of the device. In use, provided the device has been fit to fit with the wound surface in close proximity, the vacuum pressure will pull the side of the wound against this opening 15a, to seal the outlet channel end.

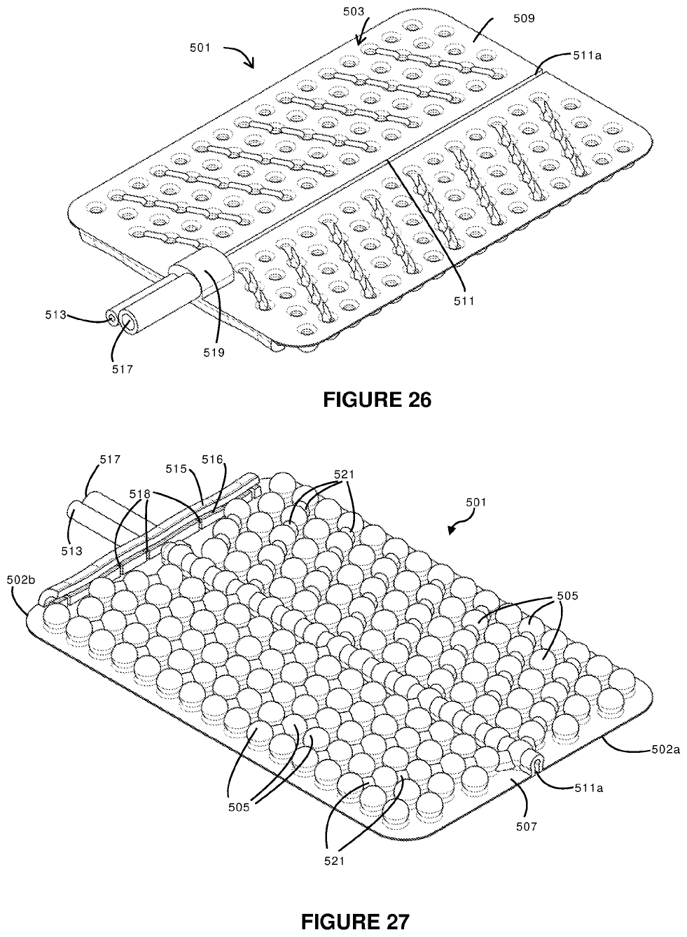

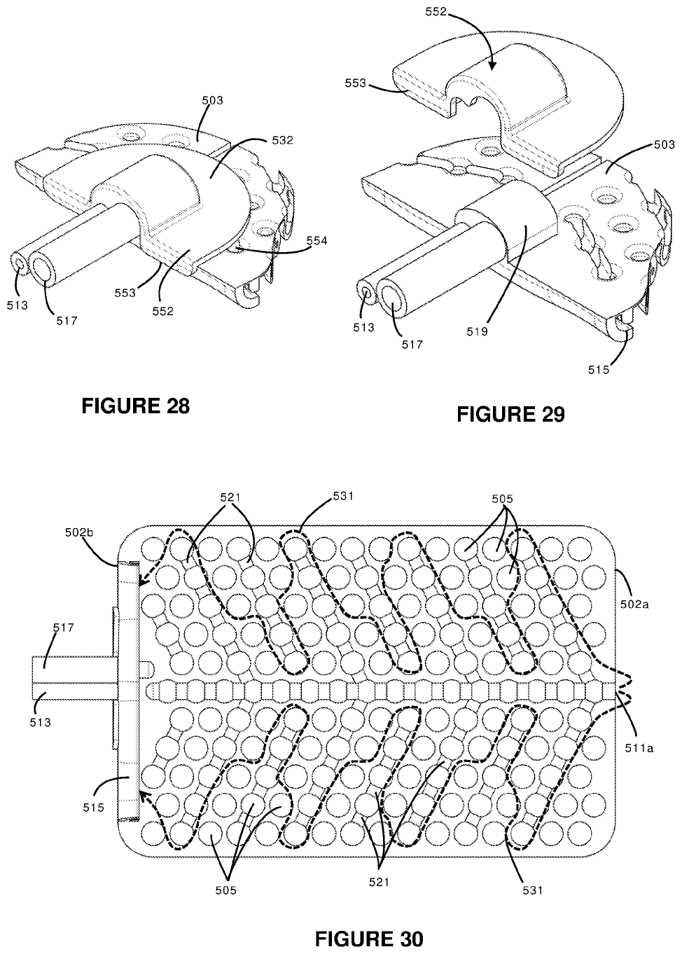

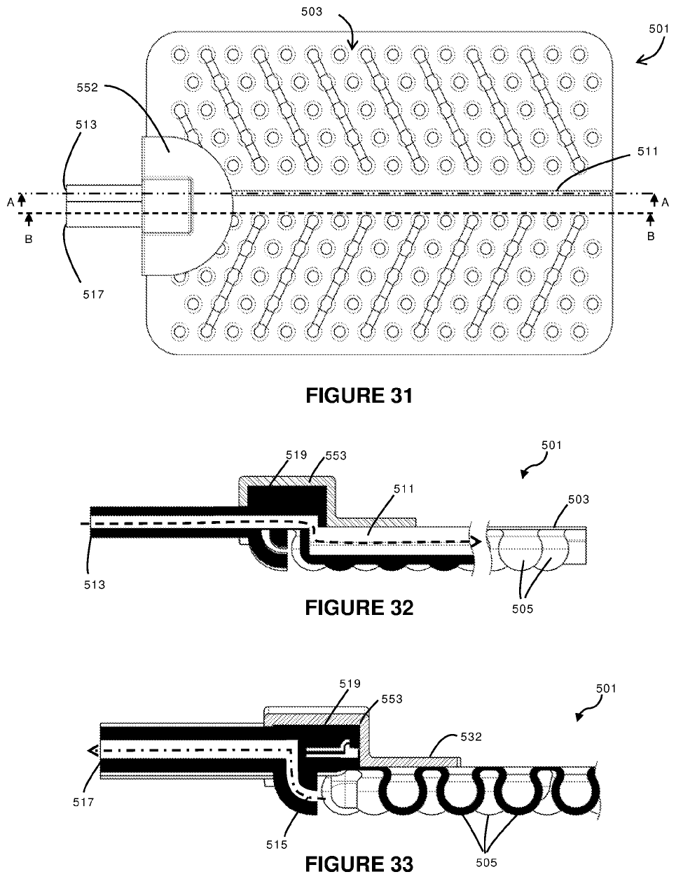

FIGS. 26 to 36 illustrate an alternative embodiment device 501 having an alternative arrangement of the inlet and outlet ports 513, 517 and an alternative spacer design 505 compared to the first embodiment device 1. Referring to FIG. 26, in this embodiment 501, the inlet and outlet ports 513, 517 are provided by short conduits that extend away from the body 503 of the device 501, in a direction substantially parallel with the plane of the body.

The orientation of the inlet and outlet conduits 513, 517 extending away from the device body 503 facilitates connection with fluid supply and removal conduits at a connection point that is away from the wound (as opposed to the connection point being directly above the wound). The connection point where the inlet and outlet ports 513, 517 couple to a respective supply or removal conduit (not shown) is a region of increased rigidity. It is advantageous to position this more rigid coupling away from the wound to minimise or avoid the likelihood of trauma from the coupling being pressed against the wound.

The inlet and outlet conduits 513, 517 are integrally moulded with the device 501 and extend from a moulded boss 519 on the top surface of the device body 503. The device 501 is preferably moulded from an elastomeric material such as silicone rubber. Internal passages within the boss 519 align with the inlet and outlet conduits 513, 517 and form respective portions of the inlet passage 511 and the outlet passage 515, in fluid communication with the respective inlet or outlet port 513, 517 as illustrated in the section views of FIGS. 32 and 33.

In use, a liquid impermeable membrane (not shown) is placed over the device 501 and adhered to the top surface 509 of the device 501. A cover clip 552 is provided to secure to impermeable membrane to the boss 519 by trapping the membrane between the cover clip 552 and the device body 503 and the boss 519. The cover clip also has a base plate 532 with a flat undersurface to press the impermeable against the device body 503 to ensure a seal is maintained. The liquid impermeable membrane may comprise an aperture with a shape corresponding to the shape of the boss 519 such that the boss extends through the aperture and the cover clip 552 presses the membrane against the surface of the device body around the boss 519 and aperture to create a seal. In some embodiments, the underside of the base plate 503a may include an adhesive to adhere to the top surface of the impermeable membrane.

The cover clip 552 is shaped to fit over the moulded boss 519, having a close fit with the boss 519 as illustrated in FIGS. 32 and 33. The cover clip 552 comprises a lip 553 and optionally one or more other features, to positively engage with one or more features on the device body 503, to releasably secure the cover clip 552 to the device body. In the embodiment shown, the lip 553 engages an undercut edge of the device 501, and resilient tabs 554 engage the hollows of the spacers 505 which are open to a top of the device 501.

In an alternative embodiment, the moulded boss 519 may include an undercut around all or a part of the circumference of the boss, preferably at the base of the boss. This undercut recess may engage with a corresponding protrusion or clip on the cover clip 552 to lock the two components together.

In this embodiment, the outlet channel comprises a transverse channel 515 that runs substantially across the width of the device 501, adjacent the second end 502b of the device 501. Referring to FIG. 27, the outlet channel 515 has a primary, elongate slit-like opening 516. The primary opening 516 opens towards the first end 502a of the deice. In addition, a plurality of spaced apart secondary slot-like openings 518 are provided along the primary opening 515. The secondary openings 518 are orthogonal to and contiguous with the elongate primary opening 516. In the event of a compressive force that presses the primary opening 516 closed, these secondary openings 518 remain open to permit fluid flow into the outlet channel 515. The channel 515 is open at its two ends, which are positioned spaced inwards from the side edges of the device 501.

The outlet channel 515 is arcuate in cross section, to direct fluid flow from the primary opening 515, substantially through 90 degrees, towards the outlet conduit 517.

The shape and arrangement of the spacers 505 in this embodiment 501 also differs from that of the first exemplified device 1. Bridges 521 are provided between some spacers 505, following a herringbone-like pattern. However, the bridges 521 are only provided in alternate spacer rows. In addition, for spacer rows that include such bridges 521, a bridge 521 to connect to the underside of the longitudinal inlet 511 is only provided in alternate ones of those rows. For the other rows having bridges, a gap is present between the underside of the longitudinal inlet 511 and the nearest spacer in said row. This arrangement may enhance fluid flow from the first end of the device 502a to the second end of the device 502b. FIG. 30 illustrates one of many possible flow paths from the end of the inlet 511a at the first end 502a of the device, towards the outlet channel 515. This flow path includes passage between said “gap” between the underside of the longitudinal inlet 511 and the nearest spacer. This spacer and bridge arrangement may also provide a greater degree of longitudinal and transverse contraction of the device when under negative pressure.

Referring particularly to FIG. 34, the wall thickness of the spacers 505 varies at different parts of each spacer. Each spacer 505 is shaped to have an ‘undercut’ region 505a adjacent to the wound facing surface 507 of the device body 503. The external surface of each spacer 505 has a convex curvature and the wall thickness of the spacer 505 narrows adjacent the device body 503 to create this ‘undercut’ 505a.

Under negative pressure, the spacers 505 are compressed causing the walls of adjacent spacers to bulge towards each other, as illustrated by the broken lines 505b in FIG. 34. The ‘undercut’ regions 505a help to control the deflection of the spacers 505 by providing a hinge point, since the spacer walls are most likely to flex about their thinnest point, which occurs at this region 505a. The undercut regions 505a thereby ensure the spacers do not defect in a way to entirely block fluid flow, and a channel 505c for fluid flow is maintained above the wound surface when the device 501 is compressed.

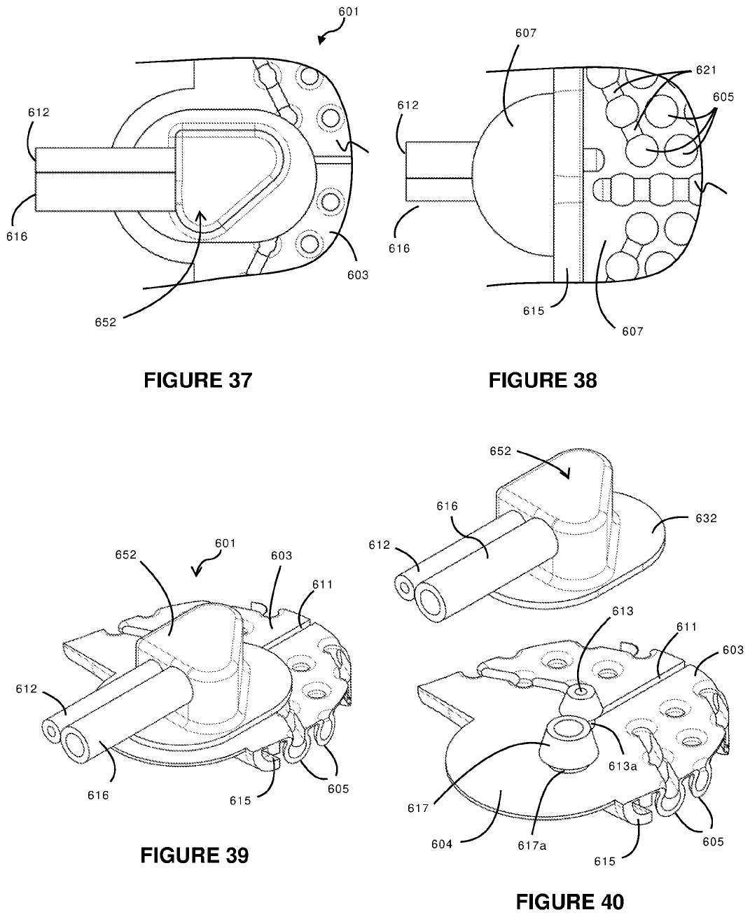

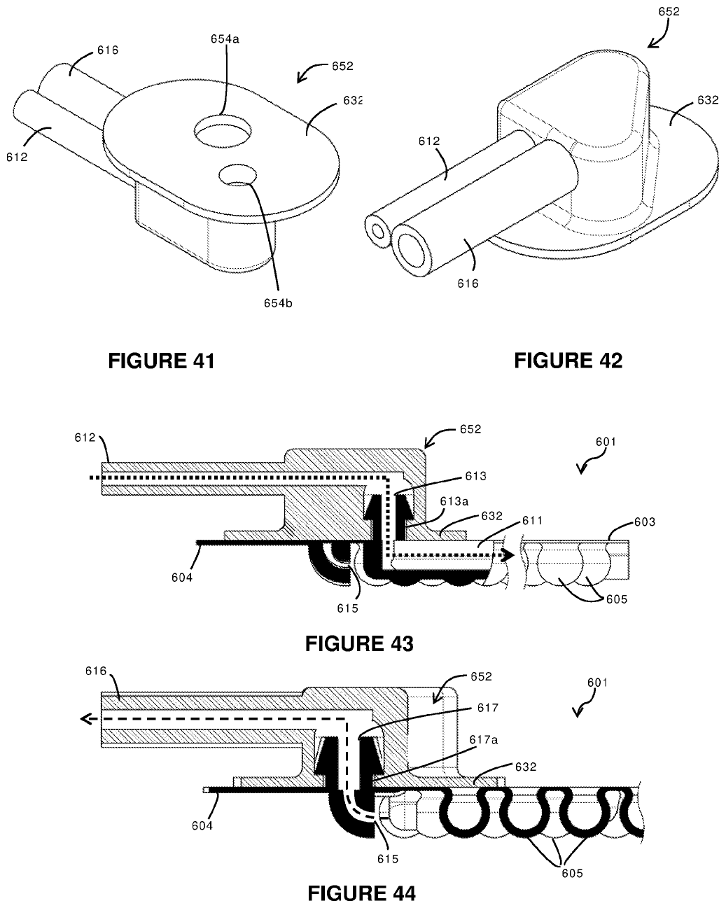

FIGS. 37 to 44 illustrate an alternative embodiment device 601 having a further alternative connector 652 and arrangement of the inlet and outlet ports 613, 617. Referring to FIGS. 39 and 40, the inlet and outlet ports 613, 617 are integral with the device body 603, and extend up from the body 603. The inlet and outlet ports 613, 617 are shaped for coupling to complementary engagement features 654 in the connector 652.

In this embodiment 601, the inlet and outlet ports 613, 617 have a mushroom-like shape, with an undercut 613a, 617b for engaging with the connector. The connector 652 comprises a base plate 632 with complementary apertures 654a, 654b for receiving the inlet and outlet ports 613, 617. The circumference of the apertures 654a, 654b each act as a lip within the respective passage, to engage with the undercut of the connectors on the body of the device.

The connector 652 comprises an inlet conduit 612 and lumen (see FIG. 43) for coupling with the inlet port 613; and an outlet conduit 616 and lumen (FIG. 44) for coupling with the outlet port 617. The connector 652 may comprise any suitable material, for example, it be moulded from a thermoplastic or elastomeric material.

The device body comprises an extension tab 604 at the second end 602b of the device 601. The extension tab 604 is shaped to accommodate the base plate 632 of the connector 652. The base plate 632 sits against the upper surface of the device 601, with part of the base plate resting on the extension tab. In use, a liquid impermeable occlusive layer (not shown) is positioned between the connector 652 and the upper surface of the device 601, the additional area provided by the extension tab 604 for interfacing with the base plate 632 of the connector, provides improved clamping between the connector 652 and device 601 for sealing the occlusive layer to the device. The liquid impermeable membrane may comprise one or two apertures shaped to allow the inlet and outlet ports 613, 617 to extend through the aperture and engage the connector 652.

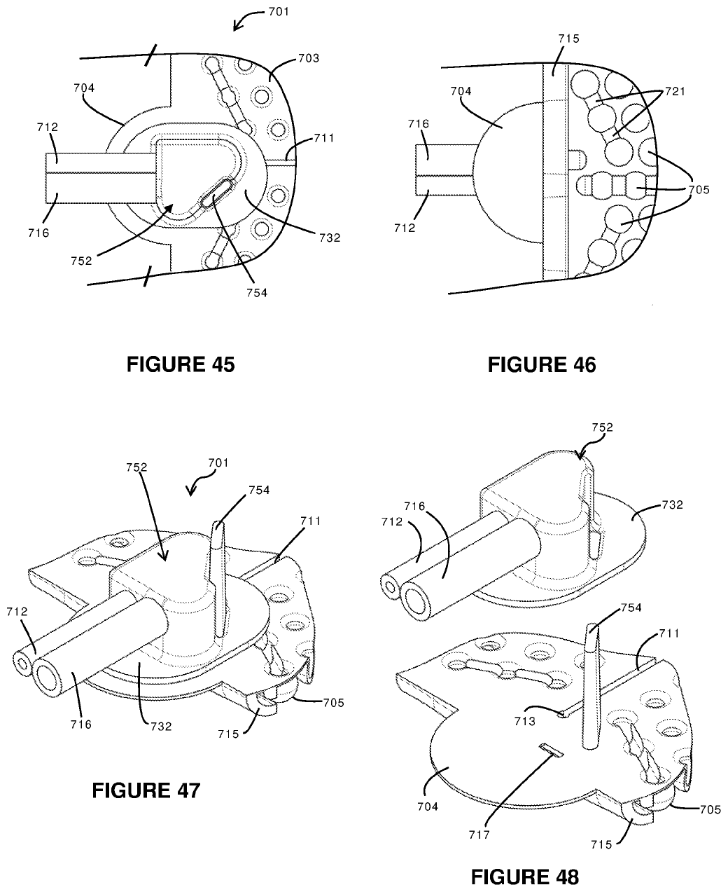

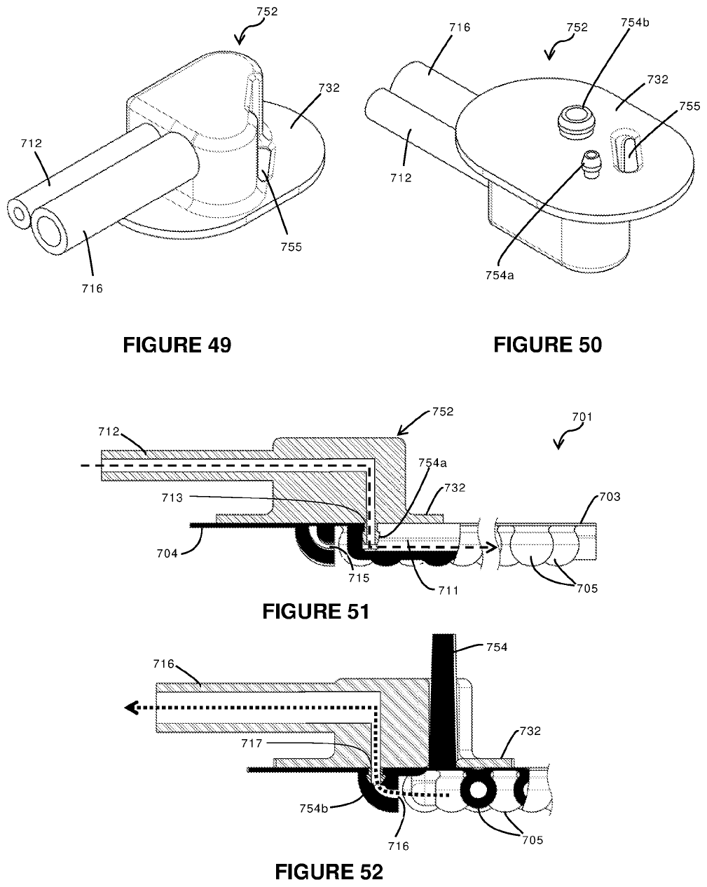

FIGS. 45 to 52 illustrate a seventh embodiment device 701 having a further alternative connector 752. Referring to FIGS. 47 and 48, the inlet and outlet ports 713, 717 in this embodiment comprise apertures in the device body 703. The inlet port 713 comprises an enlarged opening at one end of the inlet channel 711. The outlet port 717 comprises an aperture in fluid communication with the outlet channel 715. In the embodiment shown, the outlet port 717 comprises a slot-like aperture.

The connector comprises inlet and outlet engagement features 754a, 754b, for engaging with the inlet and outlet ports 713, 717. The inlet and outlet engagement features 754a, 754b comprise mushroom-like protrusions that extend from a base plate 732 of the connector 752, for receipt by the respective inlet or outlet port apertures. The protrusions have an undercut to provide a surface for engaging with the respective inlet or outlet port aperture.

The connector 752 comprises an inlet conduit 712 and lumen (see FIG. 51) for fluid communication with the inlet port 713; and an outlet conduit 716 and lumen (FIG. 52) for fluid communication with the outlet port 717.

The device 701 includes a locating tab 754 that protrudes from the top surface of the device. The locating tab 754 is configured to be received by a complementary locating aperture 755 on the connector. The tab 754 assists to ensure the connector is correctly orientated when assembled with the device body 701, to ensure the protruding inlet and outlet engagement features 754a, 754b of the connector 752 engage with the corresponding inlet and outlet ports of the device.

The locating tab 754 may also assist with assembly by helping the user to pull the device 701 towards and into engagement with the connector.

In use, a liquid impermeable occlusive layer (not shown) is clamped between the base plate 732 of the connector 752 and the upper surface of the device 701, including the additional area provided by the extension tab 704, to form an airtight seal. The liquid impermeable membrane may comprise one or more apertures shaped to allow the inlet and outlet ports 713, 717 and the tab 754 to extend through the occlusive layer.

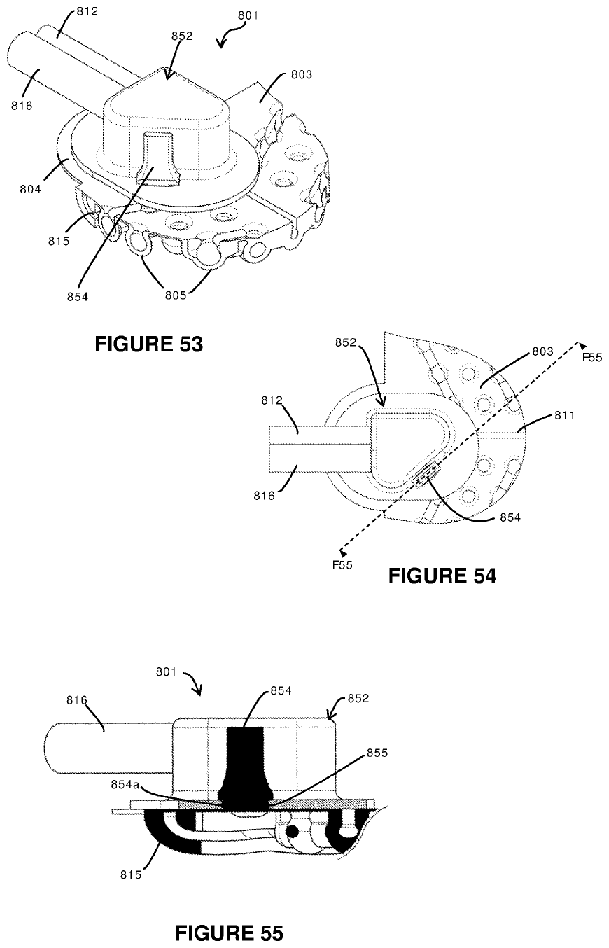

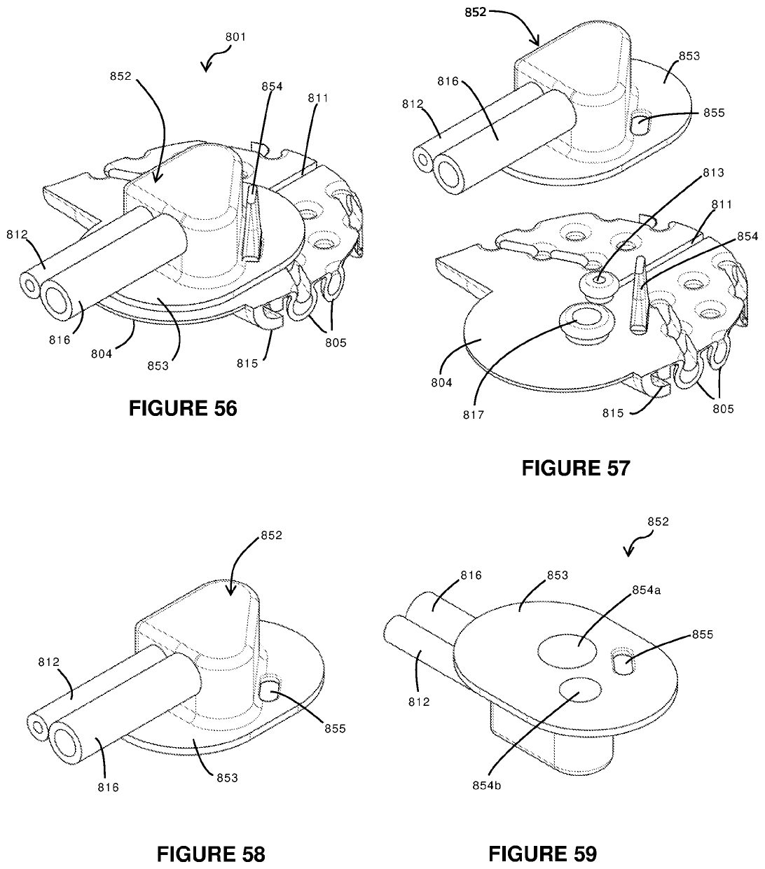

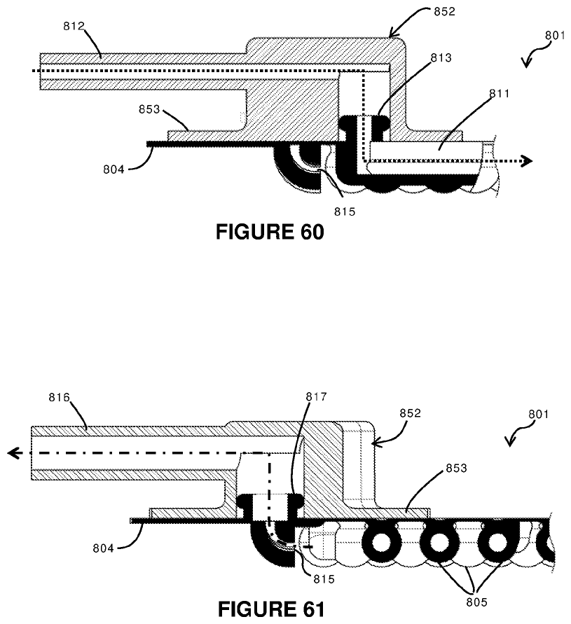

FIGS. 53 to 61 illustrate a further embodiment device 801. This device 801 is similar to the device 601 described above, but further includes a retention tab 854. The retention tab 854 protrudes from the top surface of the device 801 for receipt by a complementary aperture 855 on the connector. The retention tab comprises a narrowed or notched portion near the base of the tab. The height of this narrowed portion substantially corresponds to the thickness of the connector base plate 853 at the receiving aperture 855. Above the narrow region 854a, the tab 854 tapers from a widest point at a shoulder directly above the narrow region 854a, to the top of the tab, which may have a smaller width than the narrow region.

To couple the connector to the device, the retention tab 854 is aligned with the receiving aperture 855, and pulled through until the narrow region is engaged with the aperture and the shoulder of the wide point of the tab rests on the base plate 853 of the connector 852. The retention tab 864 is integrally moulded with the body of the device 801 and preferably comprises an elastomeric material. The parts of the tab that are wider than the receiving aperture 855 deflect as they are pulled through the aperture. The retention tab 854 assists to ensure the connector is correctly orientated when assembled with the device body 801, and also acts to lock the connector into place.

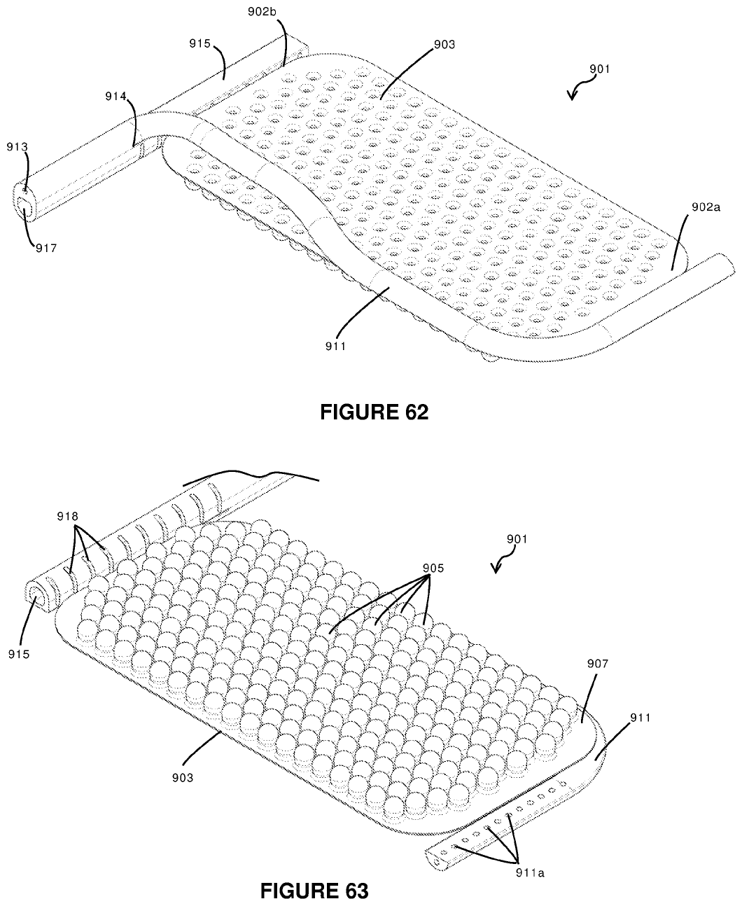

FIGS. 62 to 67 illustrate a further alternative embodiment device 901. In this device, the body 903 and the inlet and outlet 911, 915 are not integrally moulded. The inlet and outlet channels 911, 915 are provided by a dual lumen tube that splits at a juncture 914 into an inlet conduit 911 that lays along a top surface of the device body 903, extending to the first end 902a of the device; and a shorter outlet conduit 915 positioned at the second end 902b of the device.

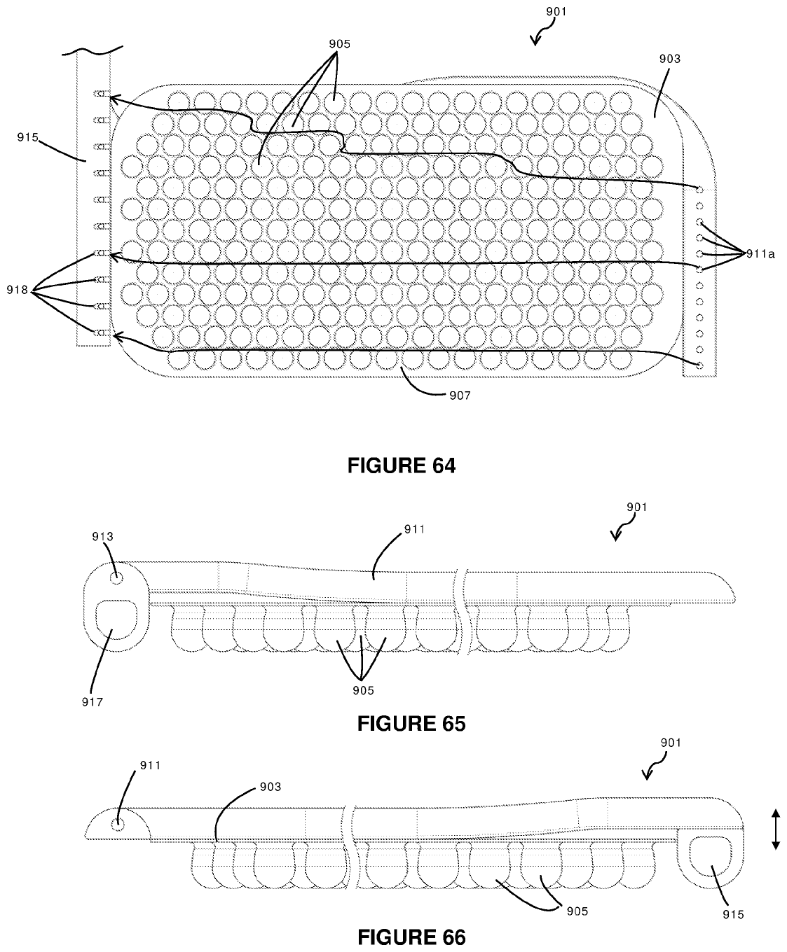

The inlet conduit 911 runs generally along one side of the device body 903, then bends to extend transversely substantially along the width of the device at its first end 902a. Referring to FIGS. 63 and 64, on the portion of the inlet conduit 911 at the first end of the device 902a, a row or apertures 911a is provided on the wound facing side of the inlet conduit 911. These apertures deliver fluid across the width of the wound. In some embodiments, these delivery apertures 911a may increase in size towards the end of the conduit for more even supply of fluids.

The outlet conduit runs substantially along the width of the device at its second end 902b. The outlet conduit comprises a transverse channel 915 provided by the hollow of the conduit and that runs substantially across the width of the device body 903. A plurality of spaced apart slot-like openings 918 are provided along the conduit 915, substantially across the width of the device body, to allow for the ingress of fluids into the conduit hollow. The secondary openings 918 are generally perpendicular to the device body 903.

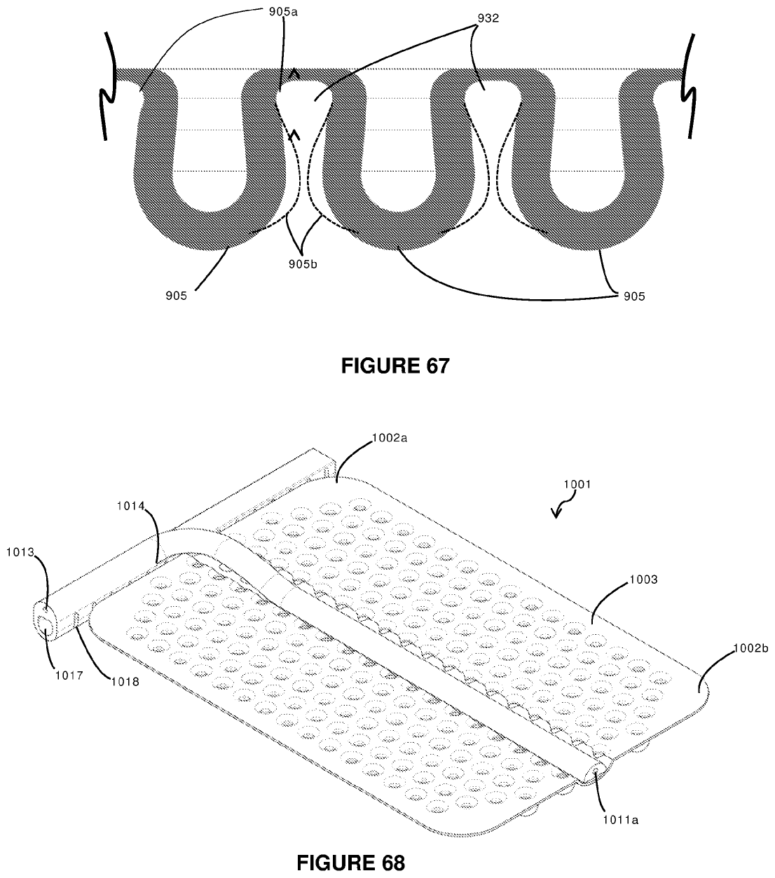

As illustrated in FIG. 67, the spacers 905 of this embodiment may be shaped to have ‘undercut’ regions 905a to ensure a fluid pathways across the device are maintained when the device is compressed in use.

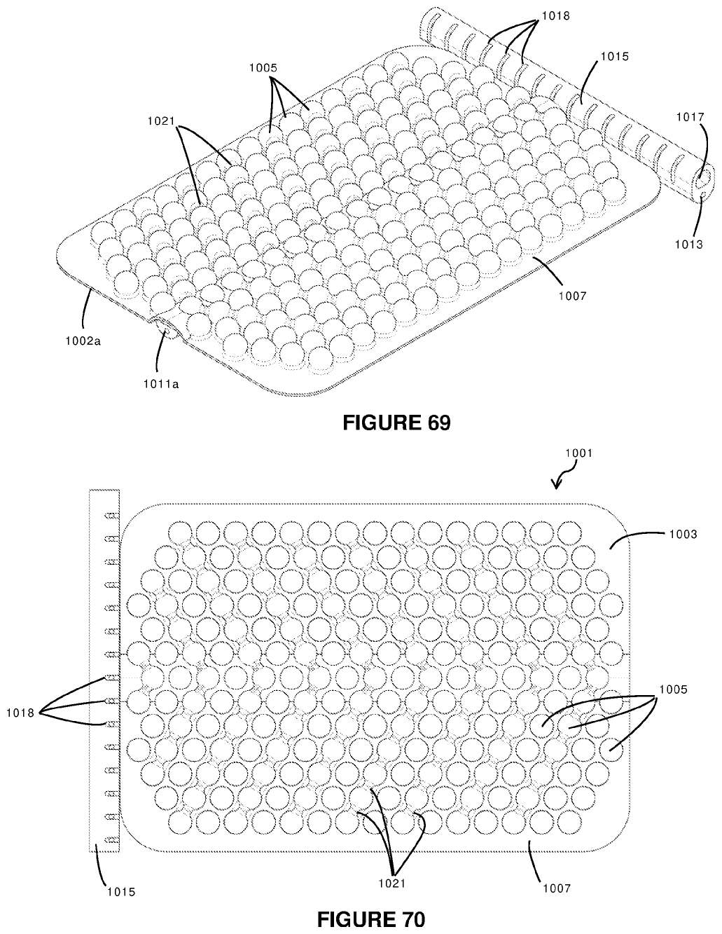

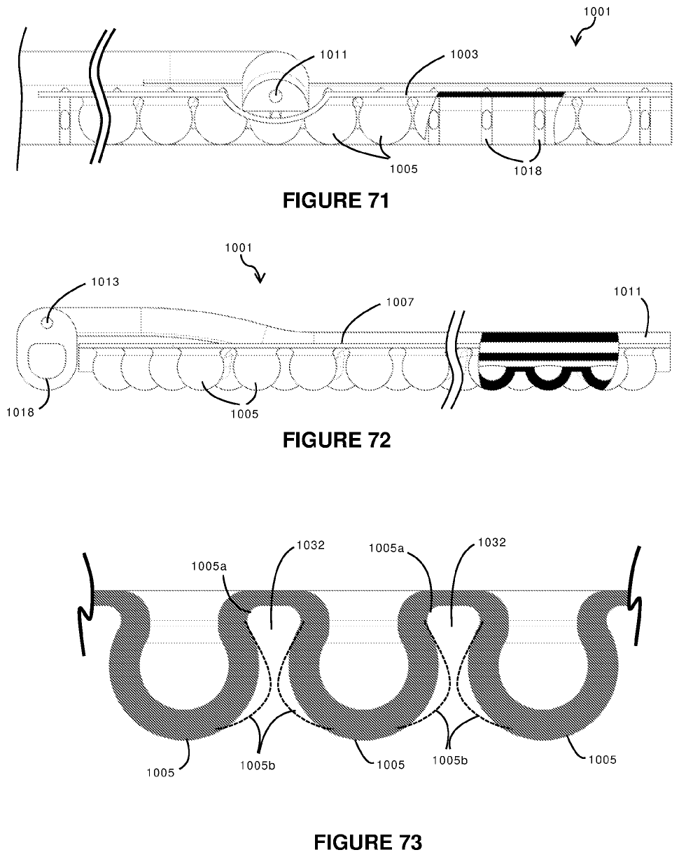

FIGS. 68 to 73 illustrate a further embodiment device 1001 that is similar to the previous device 901. However, in this device, the juncture 1014 where inlet conduit 1011 splits from the dual lumen conduit occurs near a mid-point of the device.

The inlet conduit 1011 lays along a top surface of the device body 1003, runs generally along a mid-point of the device body 1003. The device body 1003 comprises a recess for receiving the inlet conduit 1011. The inlet conduit 1011 terminates at the first end 1002a of the device to supply fluid to that end point. Some bridges 1021 are provided between adjacent spacers 1005 to assist with dispersing fluid across the wound from the single central supply point 1011a. In this embodiment, the bridges are solid, but alternatively they may be hollow.

As for the previous embodiment, the outlet conduit 1015 runs substantially along the width of the device at its second end 1002b and includes a plurality of spaced apart slot-like openings 1018 substantially across the width of the device body, to allow for the ingress of fluids into the conduit hollow.

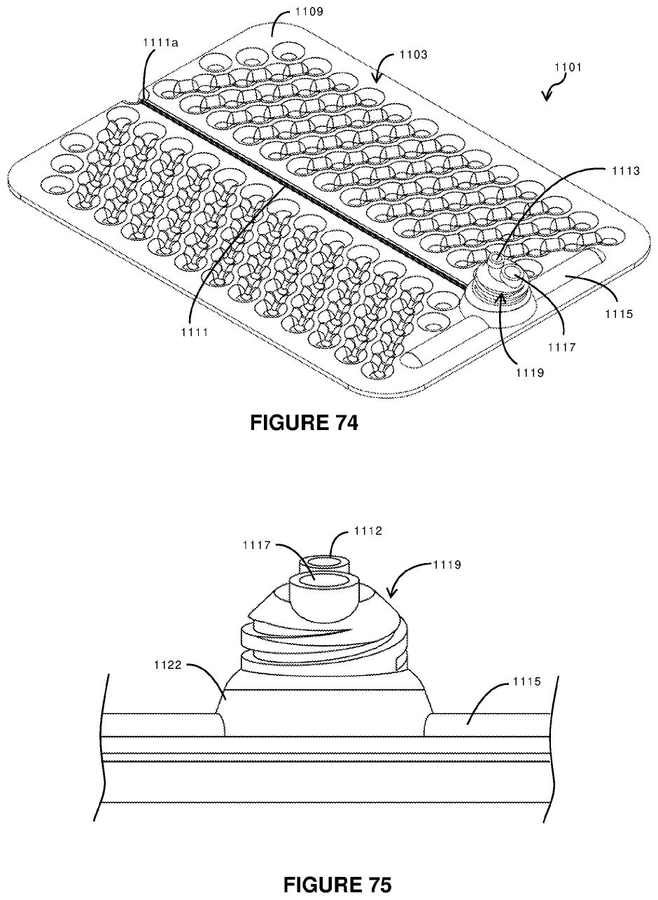

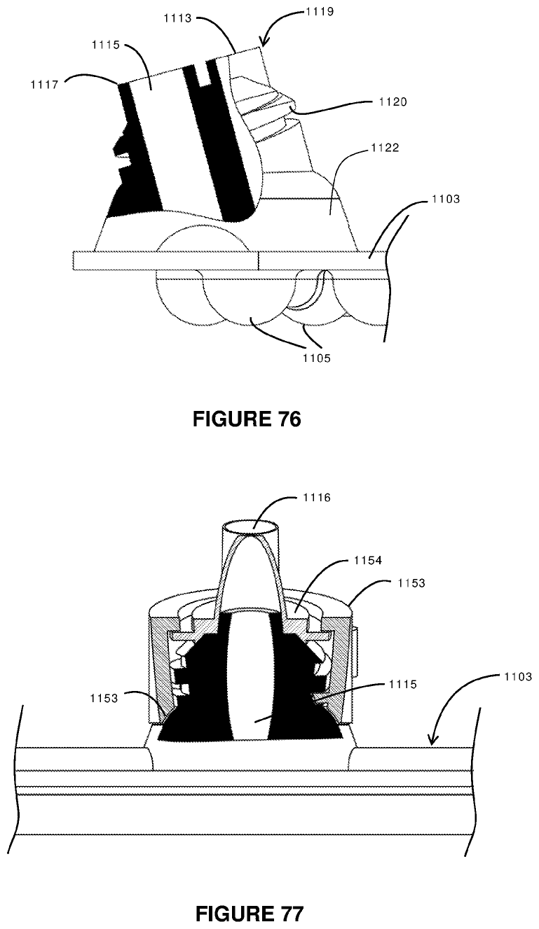

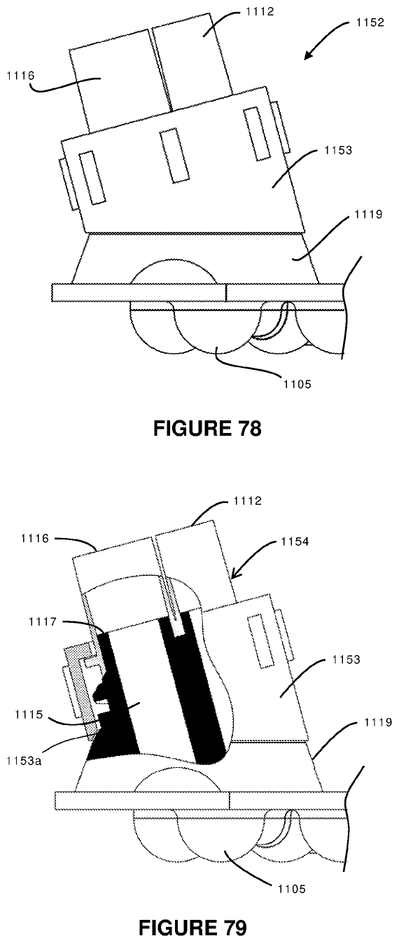

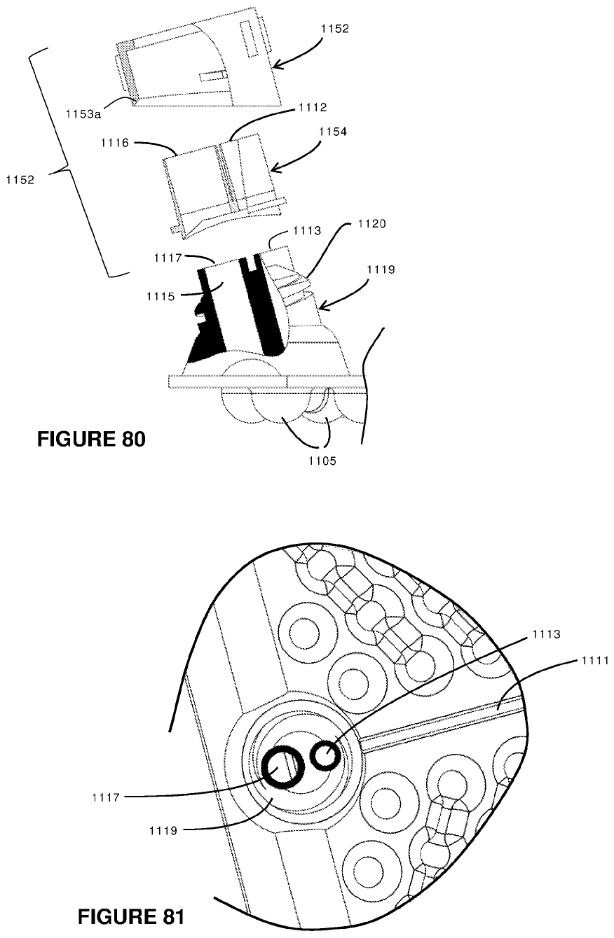

FIGS. 74 to 81 illustrate a further embodiment device 1101 having a further alternative inlet and outlet port 1113, 1117 and connector 1152 arrangement. Similar to embodiments 601, 701, 801, the inlet and outlet ports 1113, 1117 are provided integrally moulded with and extending up from the device body 1103. However, rather than the port including an engagement feature for coupling to a connector, the inlet an outlet ports 1113, 1117 extend from a boss 1119, and an engagement feature 1120 is provided on the boss.

The boss 1119 includes internal lumens defining portions of the respective inlet and outlet channels 1111, 1115 and fluidly connected to the inlet and outlet ports 1113, 1117 extending from the boss 1119. The boss 1119 comprises a threaded region 1120 for engaging with a complementary threaded connector 1152. FIGS. 77 to 81 illustrate engagement of the boss 1119 with a connector 1152. In this embodiment, the connector 1152 is a two-part assembly comprising an inlet and outlet coupling portion 1154, and a collar 1153.

The inlet and outlet coupling portion 1154 comprises and inlet and outlet limb 1112, 1116 that align with and fluidly couple to the inlet and outlet ports 1113, 1117. These limbs 1112, 1116 are configured for connection to fluid supply and removal/pressure source conduits.

The collar 1153 has a threaded internal surface for engaging with the thread on the boss. The collar is shaped to fit over the inlet and outlet coupling portion 1154 to hold it securely in place, ensuring a seal between the inlet and outlet ports 1113, 1117 and the coupling portion 1154. The collar 1153 further includes a chamfered section 1153a shaped to interface with a complementary chamfered surface 1122 on the boss 1119. These closely fitting chamfered surfaces act to seal the liquid impermeable occlusive layer (not shown) against the moulded boss 1119.

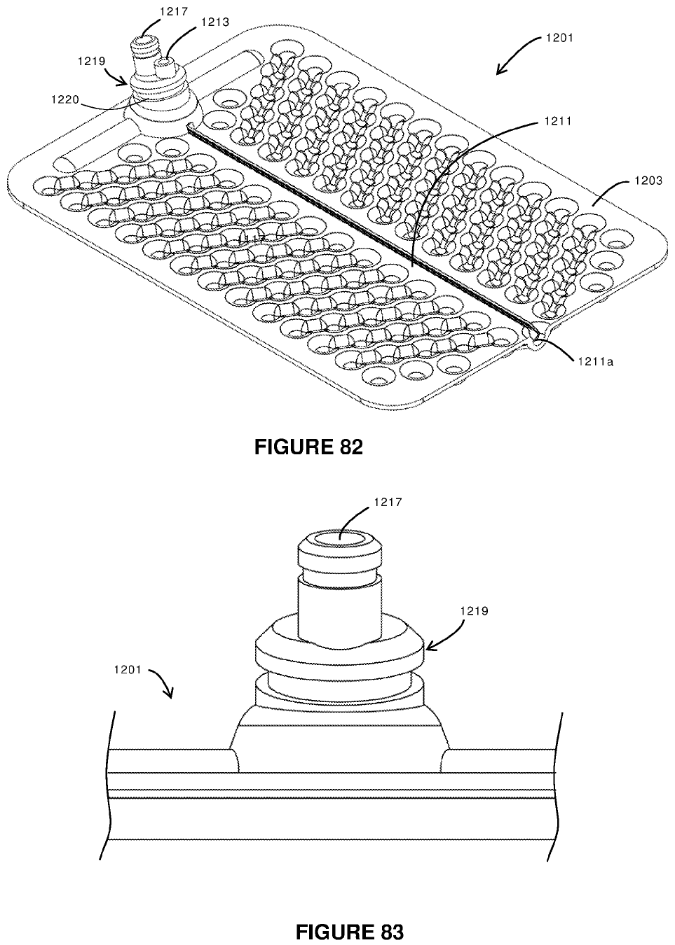

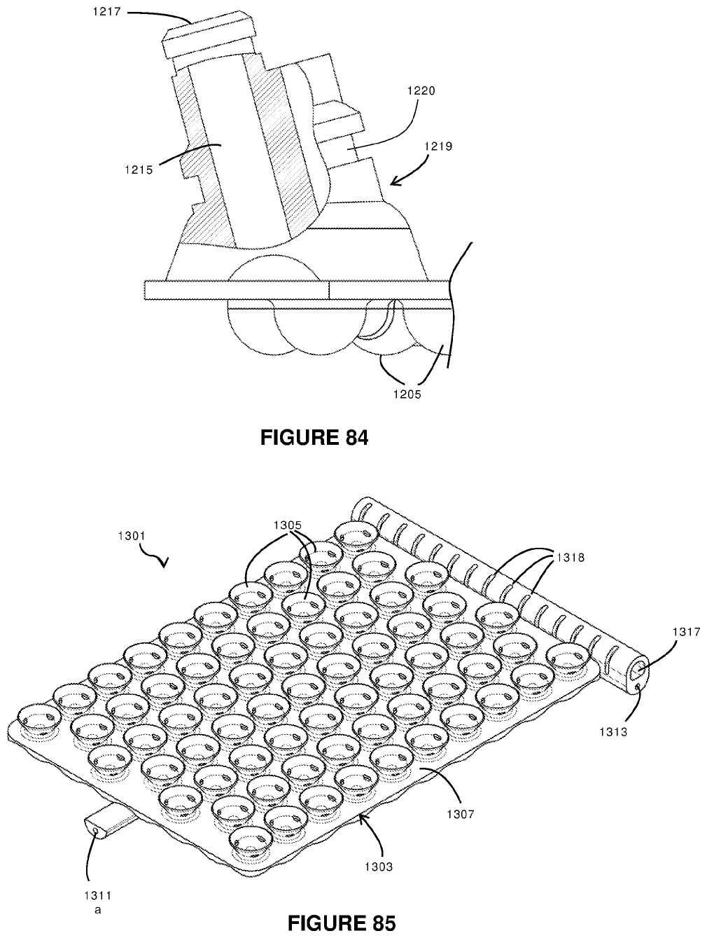

FIGS. 82 to 84 illustrate a further embodiment device 1201 similar to the previous embodiment device 1101, but wherein the boss 1219 includes an annular recess 1220 for engaging with a connector (not shown) rather than a thread. The outlet port 1217 likewise includes an annular recess and chamfered surface for engaging with a connector or downstream conduit.

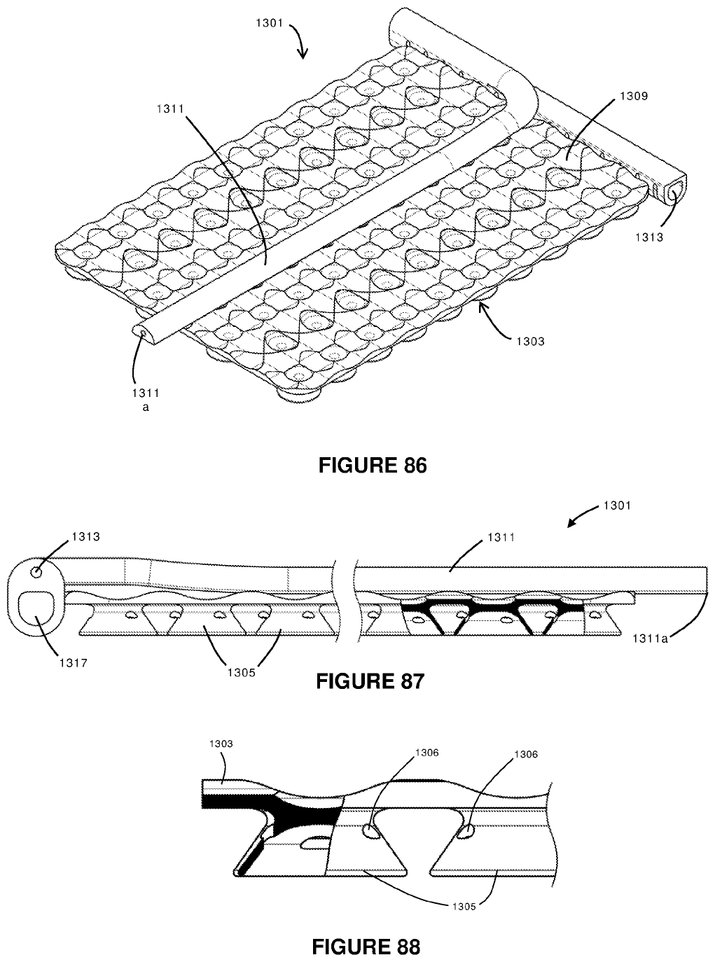

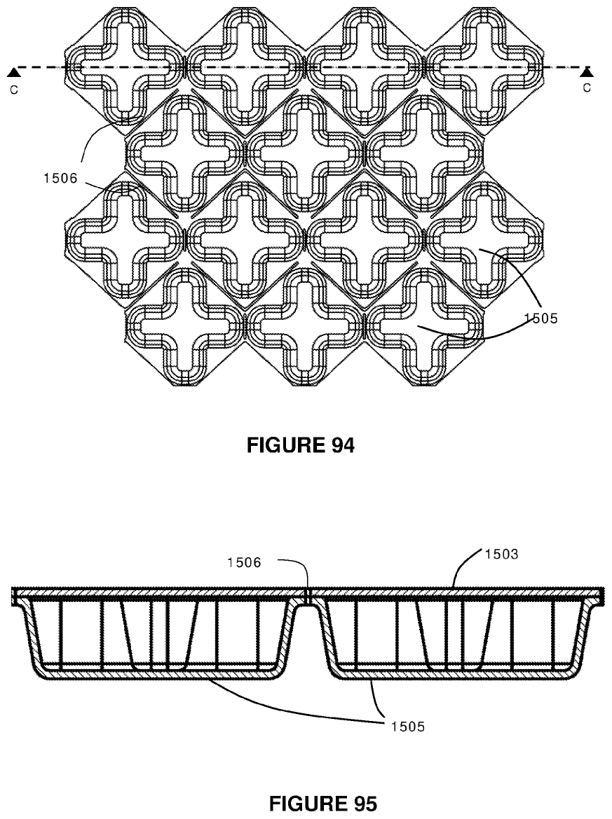

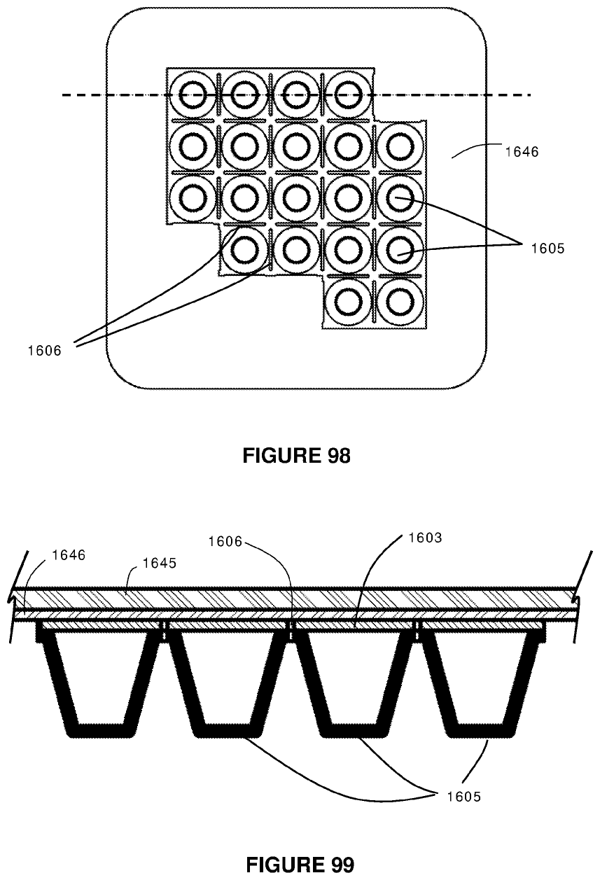

FIGS. 85 to 99 illustrate alternative embodiment spacers. In the previous embodiments, the spacers were bulbous projections having a convex lower surface, and open to the top of the device. However, alternative shapes are envisaged.

FIGS. 85 to 88 illustrate an embodiment 1301 with a cup-like spacer 1305 which has straight side walls and a wound facing concave surface. Each spacer 1305 includes one or more apertures 1306 in its side walls to permit the application of negative pressure to the wound surface below that spacer 1305, as well as fluid flow from that wound surface, through the wall of the spacer and towards the outlet 1815.

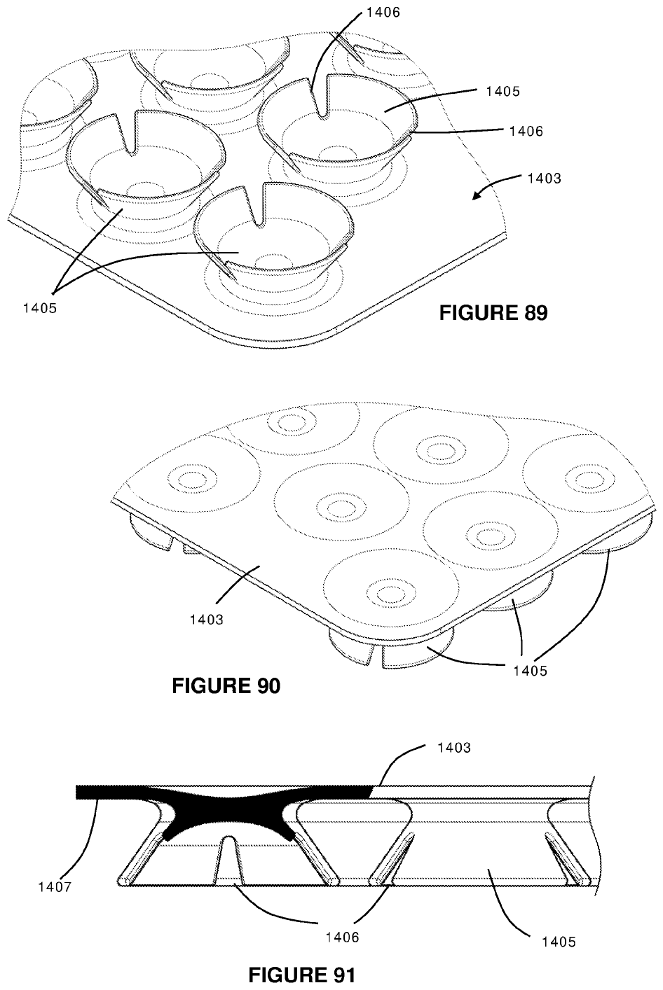

Rather than apertures 1306, cup-like spacers 1405 may have slits 1406 as illustrated in FIGS. 89-91, or any other suitable opening that permits fluid flow through the spacer walls.

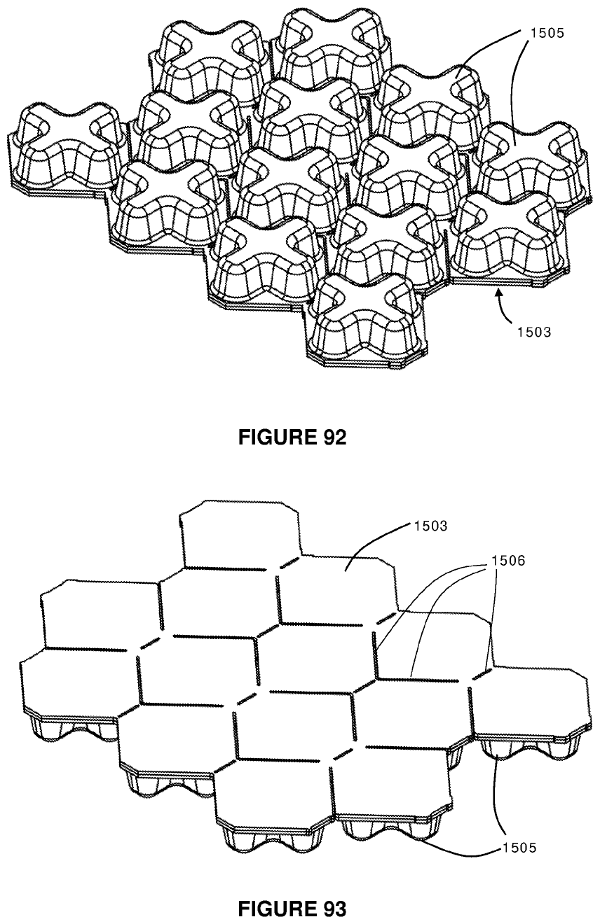

In some embodiments, the spacers may be closed on the top surface and define a sealed volume, as illustrated in the embodiments of FIG. 92 to 99. In these embodiments, the spacers 1505, 1605 are formed from a first sheet, which is joined to a top sheet 1503, 1603 at webs between adjacent spacers 1505, 1605 in a similar manner to ‘bubble wrap’. The sheets 1503, 1505, 1603, 1605 may be joined by welding or using any other suitable joining method.

In these embodiments, the spacers 1505, 1605 have a uniform wall thickness.

Perforations 1506, 1606 may be provided at the webs between adjacent spacers to allow spacers 1505 to be torn from the device to customise the size and shape of the device to thereby accommodate various wound shapes.

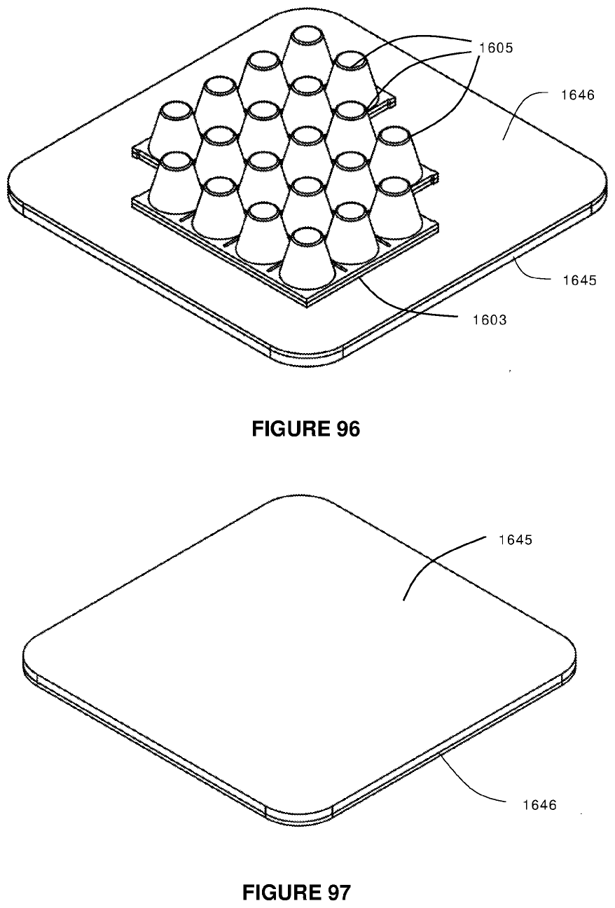

Optionally, and as shown for the embodiment of FIGS. 96 to 99, a liquid impermeable occlusive layer 1645 having an adhesive layer 1646, may be adhered to the body 1603 of the device.

### Use of Device

Referring again to the embodiment of FIGS. 5 to 15, FIGS. 16 to 21 illustrate two examples of the installation of the device 1 installed at a wound site. It will be understood that the other embodiments described herein may be utilised in a similar manner. The trimmed device 25 is placed at a wound site, with the inlet and outlet ports 13, 17 extending proud of the wound. A liquid impermeable occlusive outer layer 45 is placed over the device 25 such that the inlet and outlet ports 13, 17 extend through an aperture 47 in the occlusive layer 45. The occlusive layer 45 is adhered to the patient's skin around the peri-wound 49 to create a sealed environment over the wound and under the occlusive layer.

An underside of the occlusive layer 45 optionally comprises an adhesive surface for removably adhering a peripheral portion of the dressing to a patient's skin to seal the wound cavity and thereby allow control of the pressure within the cavity. The occlusive layer 45 is substantially liquid impermeable and substantially air impermeable. Preferably, the occlusive layer 45 has a high water vapour transmission rate (WVTR), also known as Moisture Vapour Transmission Rate (MVTR), to provide a sealed environment for the application of negative pressure but to allow moisture to exchange through the dressing. This prolongs the life of the adhesive of the occlusive layer while also helping to prevent maceration of the intact peri-wound and allowing excess fluid and exudate to vent out of the wound environment.

The occlusive layer 45 may be adhered to the top surface 9 of the device 1, 25 such that it closes or substantially encloses the top of the inlet channel 11, creating a tube-like conduit to the terminating end or opening 11a of the inlet channel.

The surface area of the occlusive layer 45 is preferably larger than that of the underlying wound therapy device 1, 25, with the peripheral portion of the occlusive layer optionally forming an adhesion flap for adhering to the peri-wound 49 to secure the dressing in place.

In some embodiments, the adhesive coating may only be applied to the underside (patient contacting side) of this adhesion flap.

In some embodiments, the occlusive layer is, for example, a transparent thin polyurethane-based sheet (for example, about 15-60 μm thick, preferably about 20 μm thick to provide good MVTR while still being easy to handle) and having a hypoallergenic 20-80 μm thick layer of silicone adhesive applied to the underside. Other types of adhesive are envisaged, for example acrylic, synthetic rubber-based, pressure sensitive adhesive, hot melt adhesive, hydrocolloid, thin absorbent skin adhesive.

The adhesive surface of the occlusive layer may alternatively be created by applying an adhesive coating to all or to a peripheral portion of the underside (patient contacting side) of the occlusive layer. Where the adhesive coating is applied to all of the underside of the occlusive layer, the occlusive layer may optionally be adhered to the top surface of the wound therapy device 1,25.

In another forms, an adhesive or seal may be separately applied around the periphery of the occlusive layer or a sealing layer may be placed over the occlusive layer so as to extend beyond the periphery of the occlusive layer to adhere and seal the wound dressing to the patient's skin.

Alternatively or additionally, to improve the liquid tightness of the seal between the dressing and the patient's skin surface, and to protect the peri-wound area, a mouldable seal/adhesive may be placed around the wound perimeter, but preferably within the boundary of the occlusive layer. To apply the dressing, the mouldable material of the mouldable seal/adhesive is optionally first applied around the peri-wound and pressed into the skin surrounding the wound, thereby filling in skin undulations and creases to act as a skin barrier and reduce fluid leakage from the dressing.

The mouldable seal/adhesive may comprise a non-curing mouldable material such as a butyl-rubber based adhesive. This allows the material to be stretched, deformed, kneaded and manipulated to create any shape whilst maintaining a high level of adhesive strength. Consequently, the mouldable seal/adhesive may be repositionable, deformable and stretchable. The mouldable seal/adhesive advantageously provides high levels of skin adhesion with a low level of trauma or pain during removal. The thickness and softness of the seal further allow the material of the seal to be depressed and moulded into skin folds and crevices for an improved seal and therefore, reduced risk of a loss of negative pressure at the wound site due to a loss of pressure

In one embodiment, the mouldable seal comprises synthetically sourced butyl-rubber which has been mixed with a tackifying resin agent, an organic filler to deaden the rubber compound into a soft and tacky singular form, and optionally a stabilising agent. In the preferred embodiment, the compound consists of Polyisobutylene, an aliphatic hydrocarbon resin as a tackifying agent, calcium carbonate as a filler material and Poly(dicyclopentadiene-co-p-cresol) as a stabilising agent. Alternatively, any suitable hypoallergenic tackifying resin could be used during the mixing and extrusion process of making the seal material, while other filler materials could include talc, dolomite, barytes, kaolin and silica. In alternative embodiments, the mouldable seal may comprise alternative mouldable adhesives or alternative rubber sources, such as mouldable polysiloxane (silicone), styrene butadiene, polychloroprene (neoprene), nitrile rubber or compounds that include blends of the aforementioned synthetic rubbers.

Release agents such as those comprising either isopropyl alcohol (IPA), hexamethyldisiloxane, 1,1,1,2-tetrafluoretan, ISOPARAFFIN L, (2-methoxymethylethoxy) propanol, Hydrotreated heavy naphtha (petroleum) or a blend of agents to the mouldable seal/adhesive 129, may be applied as required to assist with the removal of the dressing.

The mouldable seal may be provided in a strip form. In some forms, the seal is provided in a strip comprising a width of approximately 10 mm, a thickness of approximately 3 mm and a length of approximately 250 mm. In some forms, the seal strip may otherwise be provided in any widths ranging from approximately 5 mm to approximately 30 mm and with a thicknesses ranging from approximately 2 mm to approximately 8 mm, and a length ranging from approximately 50 mm to approximately 400 mm. In some forms, the mouldable seal may be provided in a roll with a total length ranging from approximately 200 mm to approximately 5000 mm. In some forms, the seal may be manually formed to a desired shape from a block of mouldable material, such as by shaping the material into an elongate, long sausage-shaped strip.

In some forms, an elongate, flat strip of the mouldable material is provided on a first removable release sheet, which is adhered to one side of the mouldable strip. A second removable release sheet is adhered to the opposite second side of the mouldable strip, such that the mouldable strip is sandwiched between the releasable sheets. In other forms the mouldable adhesive is provided in a roll with release sheets attached to both sides of the overlapping surfaces of the roll.

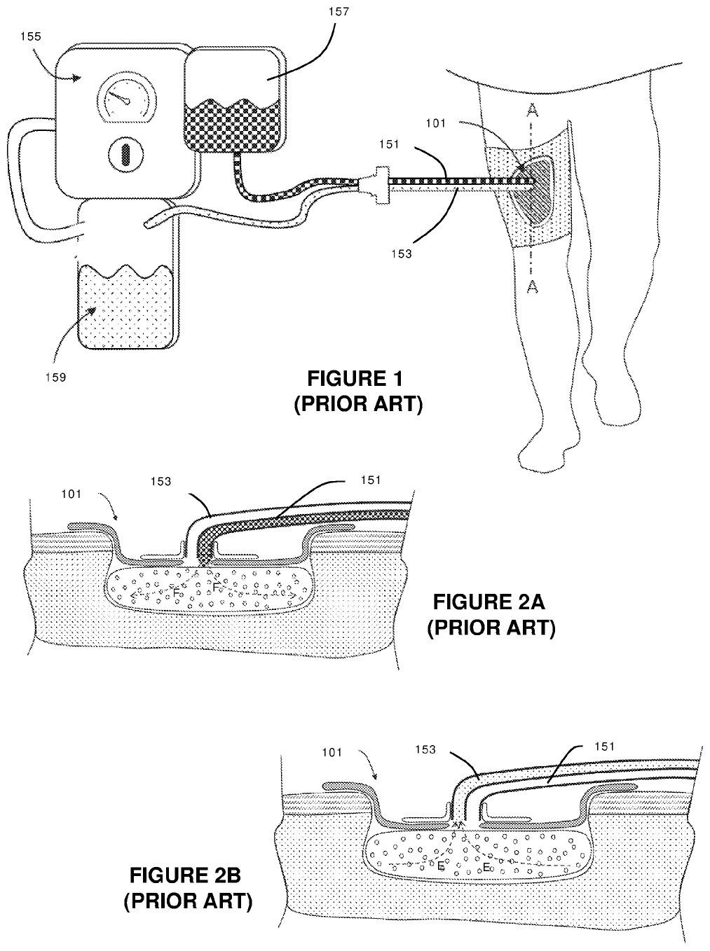

An inlet conduit 51 is coupled to the inlet port 13 at a first end of the conduit for the supply of fluids to the wound, and an outlet conduit 53 is coupled to the outlet port 17 at a first end of the conduit for the application of negative pressure and the removal of fluids from the wound (see FIG. 1).

The inlet and/or outlet conduits 51, 53 may each comprise a flexible tube, for example a plastic or elastomeric walled tube. The tube may have a wall thickness sufficient to avoid the walls collapsing under the applied negative pressure, for example, 50-250 mmHg, or up to 650 mmHg. Suitable conduits utilised for wound therapy purposes will be apparent to those skilled in the art. Alternatively, the conduit may comprise a thin wall supported by a bracing coil or other material or structure to prevent the walls of the tube collapsing under negative pressure. For example, the conduit may comprise a tube comprising a membrane or thin wall surrounding a resilient coil or an open cell foam or three-dimensional fabric or matrix.

The inlet and outlet conduits 51, 53 may be provided by separate conduits or by a dual lumen conduit having a primary lumen to couple to the outlet port 17 to supply negative pressure to the dressing, and a secondary lumen to couple to the inlet port 13. Optionally the secondary lumen or a further, tertiary lumen may be utilised to enable measurement and monitoring of the pressure within the dressing. Alternative embodiments may instead comprise a separate conduit to monitor pressure across the site.

The inlet and/or outlet conduits 51, 53 may comprise an attachment sleeve or collar 52 for attaching to the wound therapy device 1, 25. In one form, the attachment sleeve or collar is a tapered collar for securing the conduits to the device by way of a press fit. The conduit(s) may be attached to the sleeve or collar by way of bonding, welding or adhering the conduit to the sleeve to form a seal between the conduit and the sleeve or collar. The collar pinches the occlusive layer 45 against device body to provide a seal the wound therapy region 43.

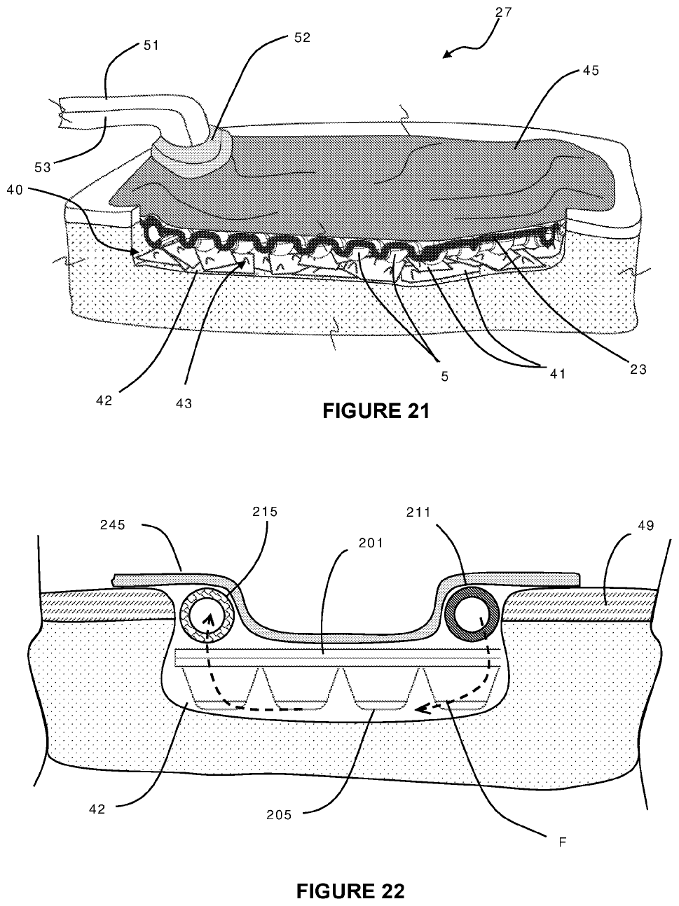

Alternatively or additionally, a mouldable material may be used to seal around the connection of the inlet and outlet conduits to the inlet and outlet ports, as shown in FIGS. 18 and 21. The mouldable material may be the butyl-rubber based material described above or could be another suitable material, for example, a two part room temperature curing silicone.

In some forms, the conduit(s) 51, 53 may be secured to the patient's skin to reduce the risk of the first ends of the conduits being inadvertently pulled from the device ports. For example, the distal end of the conduits may be secured using a piece of adhesive tape placed over the conduit and adhered to the skin.

With reference to FIG. 4, a second end 53b of the outlet conduit 53 is configured for attachment to a source of negative pressure such as a pump 55 or other common negative pressure wound therapy system. For example, the outlet conduit may have an end coupling such as a luer connector or threaded connector for attaching to a negative pressure source. Alternatively, the conduit may be sized to receive or to be received by a suitable connector as would be apparent to a skilled person. One particularly suitable pump is described in U.S. application 63/117,995, incorporated herein by reference.

A second end 51b of the inlet conduit 51 is configured for fluidly coupling to a fluid source such as a fluid reservoir 57 containing treatment fluids for delivery to the wound site. The inlet conduit may be connected to the reservoir indirectly, for example, via a pump or one or more valves, as illustrated in FIG. 1. The inlet conduit may have an end coupling to the fluid source, such as a luer connector or threaded connector. Alternatively, the conduit may be sized to receive or to be received by a suitable connector as would be apparent to a skilled person.

FIGS. 4, 7, 14 and 15 illustrate the admission and removal of fluids to and from a wound site. Treatment fluid from the first fluid reservoir 57 is delivered to the wound site via the inlet port 13 and inlet channel 11 to the first end 2a of the device 1, 25, as illustrated by the arrows F. Upon the application of negative pressure to the dressing 27, the treatment fluid and any exudate is drawn across the wound surface while the fluid is being supplied to the device, from the first end 2a of the device towards the second end 2b of the device, into the outlet channel 15 via the outlet channel opening 16, and out the outlet port 17 to the outlet conduit 53, as illustrated by the arrows E.

The system further comprises a collection reservoir 59 for collecting exudate removed from the dressing. The reservoir 59 is preferably provided downstream of the pump 55, such that the pump 55 is fluidly positioned between the collection reservoir and the device 1, 25. The fluid collection reservoir 59 is configured to include one or more air permeable filters or vents 60 to maintain the fluid collection reservoir 59 and connected conduit 61 at an ambient pressure level. The collection reservoir 59 preferably comprises a flexible bag but, alternatively, an appropriately configured rigid reservoir could be provided.

A high salt blood compatible sodium polyacrylate polymer, or other equivalent blood compatible superabsorbent polymers may be added to the collection reservoir to solidify any blood and wound fluid in the bag. These polymers are available either as loose particles, particles suspended within a dissolvable PVA film pouch or polymer suspended within a textile/fabric like medium. The use of this polymer in tandem with one or more vents on the bag avoids bag inflation and allows the fluid path of the treatment system to cope with much more air as it is introduced into the system.

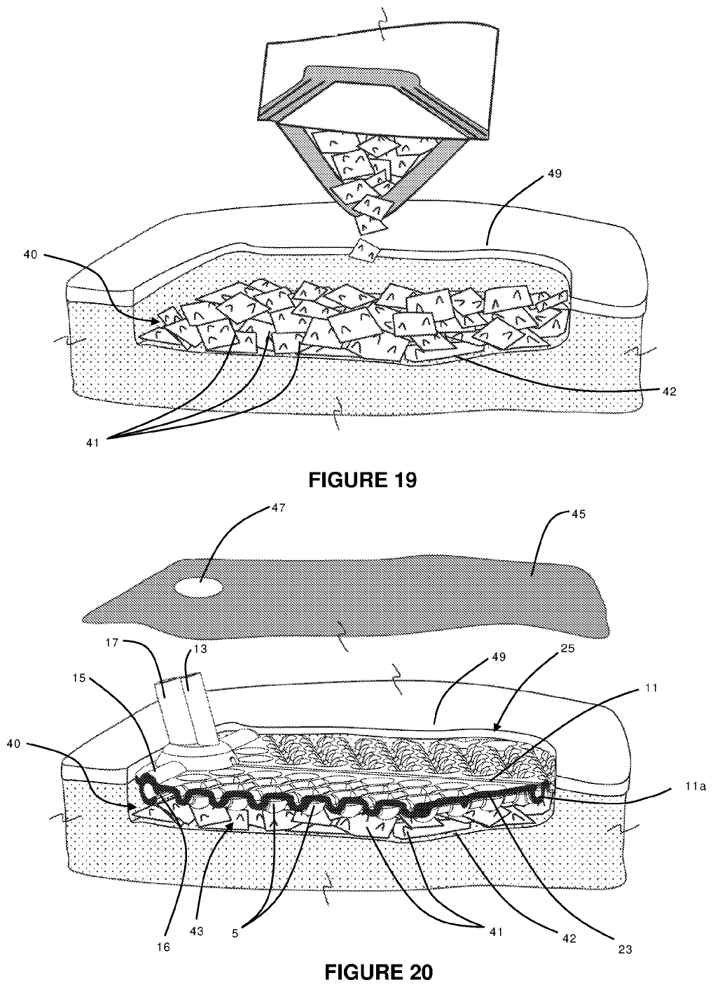

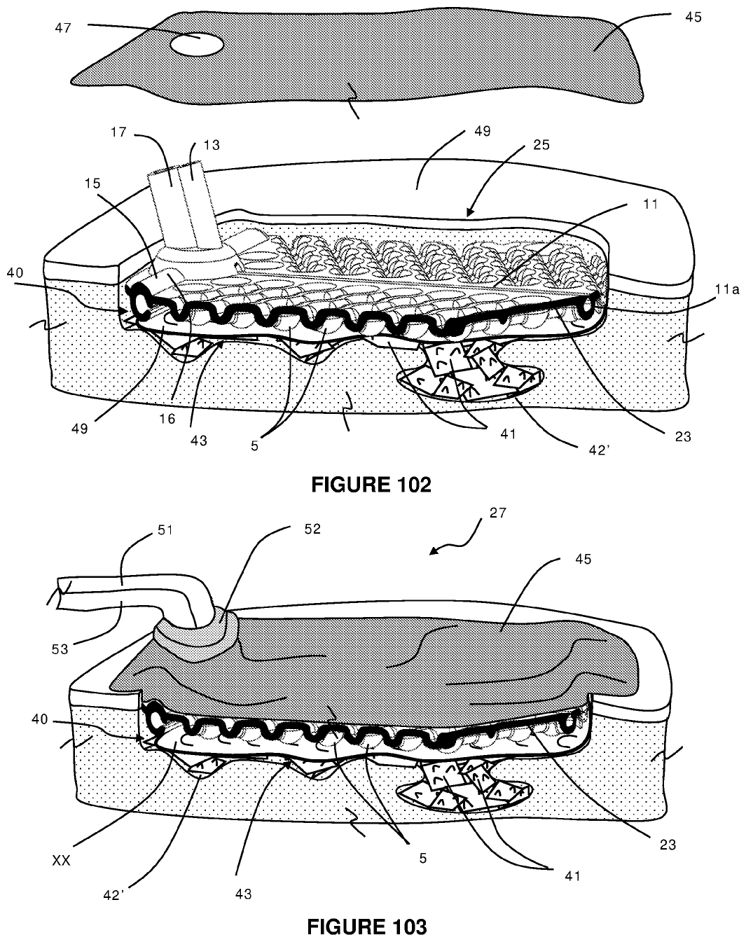

In some embodiments, as shown in FIGS. 19 to 21 and 100 to 103, the system may further include a bioresorbable layer 40 positioned between the wound interface device and the wound surface such that the spacers 5 contact the bioresorbable layer 40. The bioresorbable layer 40 is fitted substantially over the wound surface in full contact with the surface and following the wound contours. The bioresorbable layer may be used to promote tissue growth in some wounds, but may be undesirable in other applications such as when the wound is infected. The bioresorbable layer 40 preferably comprises a multiplicity of fluid paths through the layer to enable wound fluids to move from the wound surface into the wound treatment space 43.

The bioresorbable layer 40 comprises extracellular matrix (ECM) or a polymeric material. ECM-derived matrices for use in embodiments of the present invention are collagen-based biodegradable matrices comprising highly conserved collagens, glycoproteins, proteoglycans and glycosaminoglycans in their natural configuration and natural concentration. One extracellular collagenous matrix for use in this invention is ECM of a warm-blooded vertebrate. ECM can be obtained from various sources, for example, gastrointestinal tissue harvested from animals raised for meat production, including pigs, cattle and sheep or other warm blooded vertebrates. Vertebrate ECM is a plentiful by-product of commercial meat production operations and is thus a low cost tissue graft material. One exemplary method of preparing ECM is described in U.S. Pat. No. 8,415,159.

In some embodiments of the invention, resorbable polymeric material may be included in the bioresorbable layer as either lug sheets, pierced sheets, and/or in another three-dimensional form. For example, meshes comprising synthetic materials such as polyglycolic acid, polylactic acid and poliglecaprone-25 are able to provide additional strength in the short-term, but will resorb in the long term. Alternatively, the polymeric material may be a natural material, or derived from a natural material, such as proteins (e.g. collagen), polysaccharides (e.g. alginate), glycoproteins or other materials.

In some embodiments, the bioresorbable layer may comprise one or more sheets of reticulum which may be produced according to the method described in PCT application PCT/NZ2009/000152, which is incorporated herein by reference. Reticulum is a propria-submucosa of the forestomach of a ruminant that possesses a unique raised ‘honeycomb’ appearance on the luminal surface of the tissue. These honeycomb features are created by a series of continuous native ridges comprised of predominately dense collagen which create an undulating and varied textured surface on the luminal face of the reticulum tissue. The abluminal surface is generally smooth in appearance following the delamination and removal of the muscle layer. While these raised ridges retain an element of elasticity, they are relatively incompressible when subjected to the negative pressure applied within wound therapy.

In other embodiments, the bioresorbable layer may comprise one or more sheets of ECM sourced from the rumen, which is another propria-submucosa of the forestomach of a ruminant and is also described within PCT application PCT/NZ2009/000152. A multi-sheet bioresorbable layer may also comprise a two or more layers of polymeric material that have been sewn or fastened together using multi-filament or monofilament absorbable or non-absorbable suture.

The bioresorbable layer may comprise a flexible multi-sheet structure comprising plurality of overlaid sheets that are mechanically interlocked to each other, for example utilising portions of one or more of the sheets to engage with one or more of the other sheets.

The multi-sheet bioresorbable layer may comprise a plurality of apertures to enable fluid flow through the layer. The apertures may comprise slits (formed from cuts made without removing material from the layer), slots (having spaced apart side edges as a result of removed material from the layer) or any other suitable form of opening such a regular or irregularly shaped opening, through the bioresorbable layer to define a multiplicity of fluid pathways through the bioresorbable layer. The apertures or slits may be substantially X-shaped, Y-shaped, C-shaped, U-shaped, or V-shaped, or may comprise round or oval or other shaped apertures, for example.

In the embodiments shown in FIGS. 19 to 21, rather than using sheets of bioresorbable material that are each shaped to be substantially the same area and shape as the wound surface, the bioresorbable layer 40 may comprise a plurality of small pieces of bioresorbable material 41, distributed in a substantially random manner to cover the wound surface. As shown, the bioresorbable layer comprises a plurality of small pieces of bioresorbable material 41. The small pieces of bioresorbable material 41 are sized such that they have width/length dimensions of between about 1 mm and 10 mm on each edge, and in one embodiment are preferably 5 mm×5 mm in size.

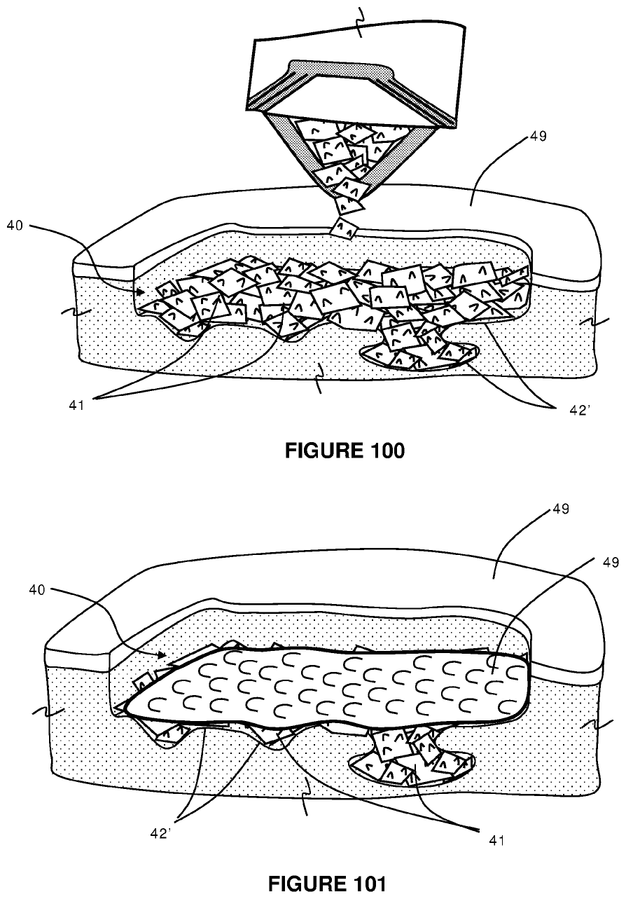

These small pieces of bioresorbable material 41 could also be used in combination with one or more sheets of ECM, with the ECM sheet(s) 49 positioned on top of the small pieces of bioresorbable material 41, for example as shown in the embodiment of FIGS. 100 to 104. As a further alternative, the small pieces of bioresorbable material could be sandwiched between two or more layers of ECM using the interlocking method described above.

The use of small pieces of bioresorbable material 41 advantageously increases the surface area of the material in contact with the wound to enable more rapid delivery of biological molecules that aid in healing, such as growth factors, proteoglycans and various proteins. The small pieces of bioresorbable material may also provide better conformity of the bioresorbable layer to the irregular contours of the wound bed, thereby also providing better transfer of the negative pressure to the wound surface. This is particularly advantageous in the common occurrence of wound surfaces 42′ having undermining, such as shown in FIGS. 100 to 103. The small pieces of bioresorbable material 41 fill the undercuts and cavities to create a more uniform surface onto which to place the device.

FIGS. 22 to 24 illustrate further examples of installation of an alternative device 201, 301, 401 installed at a wound site 42. In this alternative device 201, 301, 401 the inlet and outlet are not integrally formed with the body of the device, instead, inlet and outlet conduits 211, 311, 411, 215, 315, 415 are provided separate to the device. These inlet and outlet conduits 211, 311, 411, 215, 315, 415 may be separate conduits to each other or may be two lumens of a dual lumen conduit that split proximal the wound site. The inlet conduit 211, 311, 411 is arranged to deliver fluids to a first end of the device, the outlet conduit 215, 315, 415 is arranged to remove fluids (and apply negative pressure at) a second end of the respective device 201, 301, 401.

FIG. 22 illustrates a relatively deep wound, where the device 201 is placed with the spacers 205 in contact with the wound surface. The device 201 is shaped and dimensioned to extend substantially the full width of the wound site. The inlet and outlet conduits 211, 215 are positioned on top of the device 201, with one at each end of the device. Fluid from the inlet 211 enters the wound treatment space 42 between the edge of the device 201 and the side of the wound, then flows towards the opposite end of the device before flowing between the opposite edge of the device 201 and the side of the wound, into the outlet 215, as illustrated by arrows F.

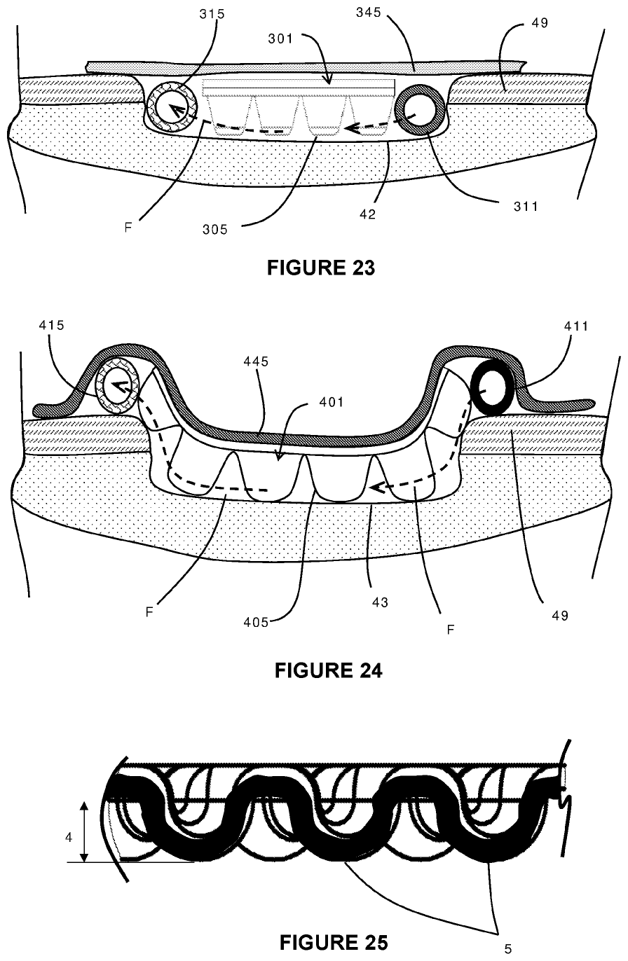

FIGS. 23 and 24 illustrate the positioning of a device 301, 401 in a shallower wound. In the embodiment of FIG. 23, the device 301 does not extend the full width of the wound, and the inlet and outlet conduits 311, 315 are positioned in line with the device 301, in contact with the wound surface, at opposite ends of the device. Fluid from the inlet 311 enters the wound treatment space 42 directly then flows towards the opposite end of the device before flowing into the outlet 315, as illustrated by arrows F.

Finally, in the embodiment of FIG. 24, the device 301 is wider than the wound width such that it extends up the sides of the wound, with the inlet and outlet conduits 411, 415 positioned on the surface of the skin, at opposite ends of the device. Fluid from the inlet 411 flows down into the wound treatment space 42 before flowing towards the opposite end of the device and up into the outlet 415, as illustrated by arrows F.

In each of the arrangements shown in FIGS. 22 to 24, a liquid impermeable occlusive outer layer 245, 345, 445 is placed over the device 201, 301, 401 with that the inlet and outlet conduits 211, 311, 411, 215, 315, 415 extending through respective apertures (not shown) in the occlusive layer. The occlusive layer 245, 345, 445 is adhered to the patient's skin around the peri-wound, and preferably to an upper surface of the device 201, 301, 401, to create a sealed environment over the wound and under the occlusive layer.

### System

For all embodiments, the inlet and outlet conduits 51, 211, 311, 411, 53, 215, 315, 415 are configured to fluidly couple to the inlet port 13 and outlet port 17 respectively, and thereby to the dressing 27. The coupling is sealed to ensure the wound is air-tight. The inlet and/or outlet conduits 51, 53 are coupled to a source of negative pressure such as a vacuum pump, and the pump operated to create a continuous, or variable (e.g. oscillating or intermittent) vacuum within the sealed dressing. The negative pressure assists the removal of fluid from the wound and may improve blood flow to improve wound healing.

The pump 55 or system 50 may include an air inlet valve upstream of the wound treatment device inlet port 13, an actuator to drive the air inlet valve between an open position and a closed position, a controller in communication with the actuator, and a motor driving the pump to operate the air inlet valve and the pump.

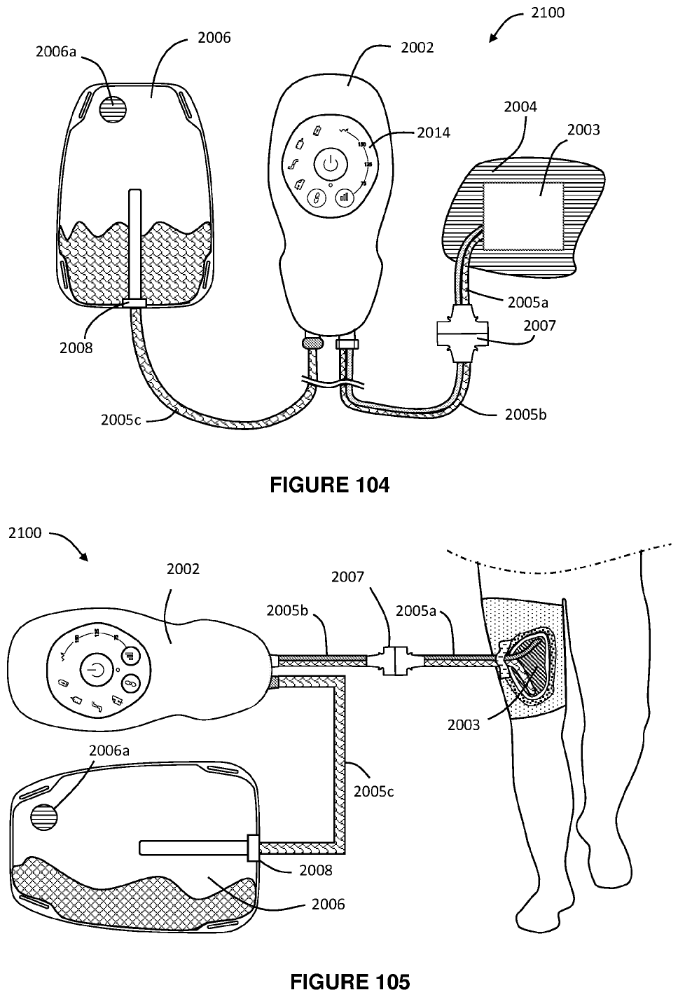

FIGS. 104 to 109 show exemplary embodiments of negative pressure treatment systems 2100, 2200, 2300 (herein treatment systems) for the removal of fluid from a wound or for supplying treatment fluid to a wound 2004 and removing fluid from a wound using a wound treatment device 2003. The wound treatment device 2003 of the system may be any one of the devices 1 . . . 1301 described herein.

In relation to the exemplary embodiment systems, like reference numbers are used for different embodiments to indicate like features.

Referring to FIG. 104, at a general level the treatment system 2100 comprises a wound treatment device 2003 to be located at a wound treatment site 2004 (‘wound’), a vacuum pressure unit 2002 comprising a vacuum pump assembly for applying negative pressure to the wound 2004 via the treatment device 2003, and a fluid collection reservoir 2006 for collecting fluid returned from the wound 2004. FIG. 105 illustrates the system 2100 of FIG. 104 used with a dressing or external wound treatment device 2003 for an open leg wound.

The vacuum pressure unit (or vacuum unit) 2002 is configured to position the pump assembly 2015 upstream of the fluid collection reservoir 2006 and downstream of the wound treatment device 2003. The fluid collection reservoir 2006 is configured to include one or more air permeable filters or vents 2006a to maintain the fluid collection reservoir 2006 and connected conduit 2005c at an ambient pressure level.

The vacuum unit 2002 fluidly couples to the wound treatment device 2003 via at least one conduit. The conduit from the vacuum unit 2002 to the wound treatment device 2003 may comprise a two-part conduit, with a first conduit 2005b extending from the vacuum unit 2002, and a second conduit 2005a extending from the wound treatment device 2003. The second conduit may be part of the wound treatment device 2003 or may be connected to the treatment device 2003 by a connector (not shown). A connector 2007 is provided to fluidly couple the first and second conduits 2005a, 2005b. Alternatively, a continuous conduit may extend between the vacuum unit 2002 and the treatment device 2003.

The connector 2007 may comprise a one-way valve oriented to allow fluid flow in a direction from the wound 2004 towards the vacuum unit 2002 and prevent a backflow of fluid from the pump to the wound. In alternative embodiments, a one-way valve may instead be provided within the vacuum unit 2002, elsewhere on the conduit 2005a, 2005b, or as part of the treatment device 2003. In a further alternative, the treatment system 2100 may be without a one-way valve between the treatment device 2003 and the vacuum unit.

In some embodiments, the conduit(s) between the vacuum unit 2002 and the treatment device 2003 may comprise a dual lumen conduit with a primary lumen for the passage of fluid flowing from the wound to the pump assembly 2015, and a secondary lumen. The secondary lumen may allow for measurement of pressure at the wound site. The secondary lumen provides for the delivery of air and/or treatment fluids to the wound 2004. However, in alternative embodiments multiple conduit(s) may be provided between the vacuum unit 2002 and the treatment device 2003 each with a single lumen.

A further conduit 2005c is provided between the vacuum unit 2002 and the reservoir 2006 to fluidly couple the pump assembly 2015 to the reservoir 2006. A connector 2008 may be provided to fluidly couple the conduit 2005c to the reservoir 2006.

In preferred embodiments, the vacuum unit 2002 is a portable hand-held unit. The vacuum unit 2002 may be a single use unit that is intended to be used for a single patient. In an alternative embodiment the vacuum unit 2002 could be configured for multi-patient use. The vacuum unit 2002 comprises a (plastic) shell or enclosure to house the pump assembly 2015 and other components. The vacuum unit 2002 comprises a user interface 2014 for operating the vacuum unit 2002. The user interface may include controls to turn the pump assembly 2015 of the system 2100 on and off, and may allow an operator to control parameters of a pressure treatment being applied to the wound 2004 such as the level of vacuum pressure being applied or the length, size and frequency of pressure oscillations between upper and lower set points.

In alternative embodiments the user interface 2014 may also include controls to remotely connect a monitoring device to the vacuum unit to enable the transmission of data to an operator or user of the system to aid in the monitoring of treatment.

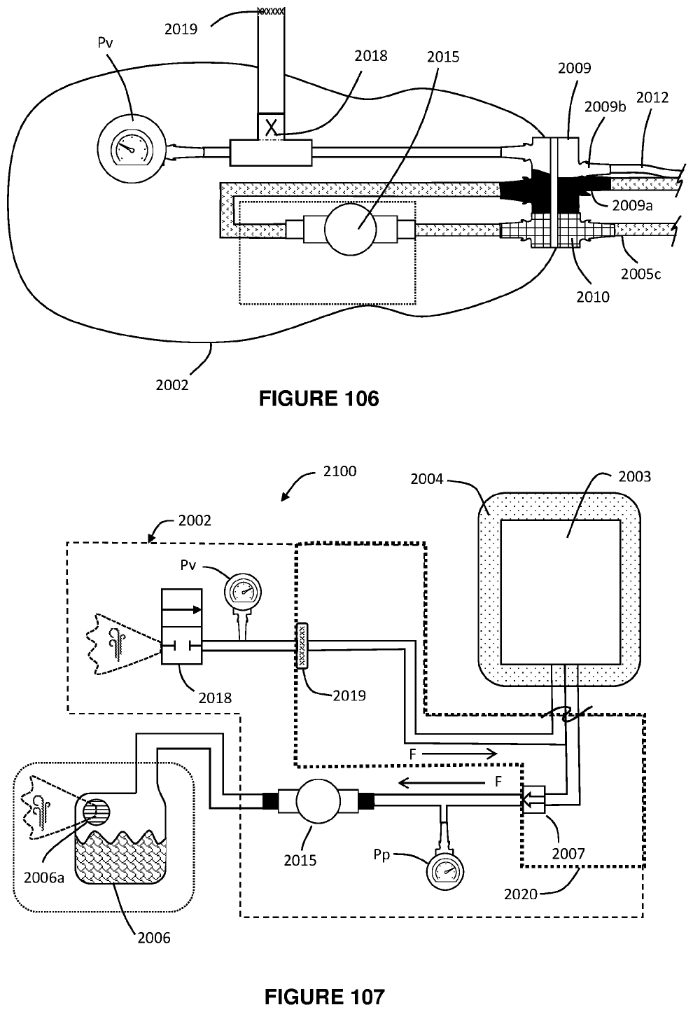

Referring now to FIG. 106 together with FIG. 104, the vacuum unit 2002 comprises a housing or enclosure that houses a vacuum pump assembly 2015 described in more detail below, batteries or other power supply, a vacuum unit connector 2009 in fluid communication with the conduit(s) 2005b, 2005a to deliver and receive fluid from the wound treatment site 2004, and a vacuum unit outlet connector 2010 in fluid communication with the conduit 2005c to the reservoir 2006, for the fluid flow from the pump assembly 2015 to the reservoir 2006. The connectors 2009, 2010 are configured to couple with ends of respective conduits 2005b, 2005c and may be of any suitable form, for example, they may comprise luer-type connectors.

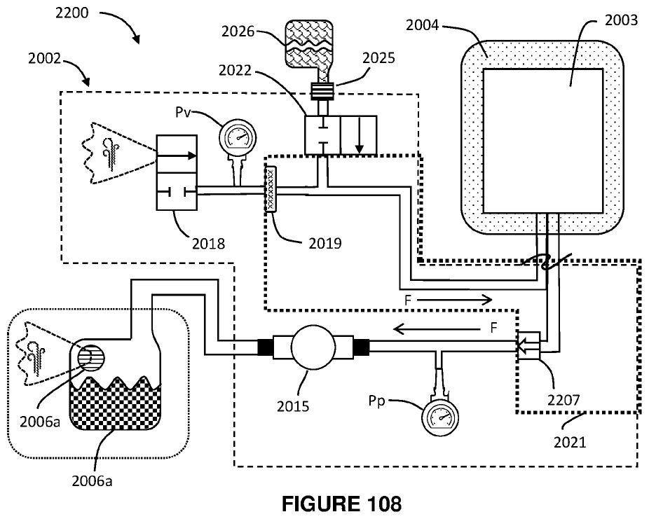

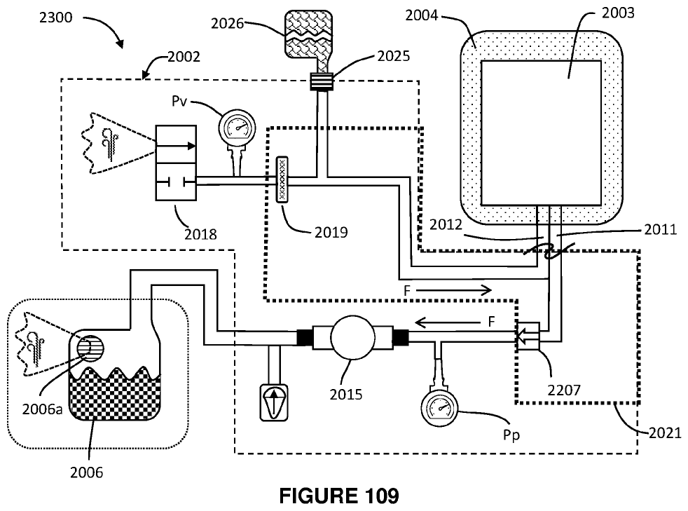

In one embodiment the vacuum unit connector 2009 may comprise two one-way valves such that a one-way valve within the secondary connector 2009b is oriented to allow the flow of fluids from an upstream source, such as ambient air that has been passed through a sterile filter (filter 2019 in FIGS. 106 and 107) or from a treatment fluid reservoir (reservoir 2026 in FIGS. 108 and 109), to the wound 2004. The corresponding one-way valve within the primary connector 2009a is oriented to allow the flow of fluid in a direction from the wound 2004 towards the vacuum unit 2002. In some embodiments the one-way valves within the primary 2009a and secondary 2009b connector may be configured to be closed when the vacuum unit connector 2009 is disconnected from the vacuum unit 2002. These valves are then subsequently opened to allow the passage of fluids when the vacuum unit connected 2009 re-connected to the vacuum unit 2002. Examples of known prior art connectors that possess such features include needle-free or needless connectors for use within IV applications, such as the BD® MaxPlus™ needle-free connectors, which only allow a passage of fluid once engaged with an appropriate leur-lock connector.

The conduit 2005b for fluid flow into and out of the vacuum unit connector 2009 is a dual lumen conduit with a primary lumen 2011 and a secondary lumen 2012. The connector 2009 includes a primary connector 2009a providing a fluid inlet to connect to the primary lumen 2011, and a secondary connector 2009b providing a fluid outlet to connect to the secondary lumen 2012 while keeping the flow from these lumens separated. The larger primary lumen 2011 allows the passage of fluid flowing from the wound, through the primary connector, to the vacuum pump assembly 2015. The secondary or supply connector 2009b may be separate from the primary or removal connector 2009a.

The primary and secondary lumens 2011, 2012 are preferably provided as adjacent passages in a single body/conduit along most of their length. However, adjacent the vacuum unit 2002 and/or adjacent the wound treatment device 2003, the dual lumen conduit 2005a, 2005b may be split or separated into two separate limbs or conduits, a supply conduit comprising the secondary lumen 2012 and a removal or exudate conduit comprising the primary lumen 2011, for ease of coupling to the vacuum unit 2002 and/or to allow the supply conduit to enter the wound or wound treatment device 2003 at a different location to the removal conduit. The primary or removal conduit and lumen may be referred to interchangeably and referenced by reference numeral 2011 and the secondary or supply conduit and lumen may be referred to interchangeably and referenced by reference numeral 2012.

The supply conduit 2012 is in fluid communication with a pressure sensor Pv to allow for measurement of pressure on an upstream side of the wound treatment device 2003.

The vacuum unit 2002 comprises an air inlet valve 2018 in fluid communication with the supply conduit 2012. The air inlet valve 2018 is controlled in a manner to introduce air into the treatment system 2100 to assist with lifting fluid from the wound site 2004, as described in more detail below.

As shown in FIG. 106, the air inlet valve 2018 may have an inlet to draw ambient air to the system from outside the vacuum unit 2002 enclosure. Alternatively, the inlet for the air inlet valve may take air from inside the vacuum unit housing/enclosure.

A sterile filter 2019 is provided to prevent the ingress of bioburden and non-sterile air into the system 2100 and wound site 2004. In FIG. 106, the filter 2019 is provided on an inlet of the air inlet valve 2018, however a filter may be placed at another location between the air inlet valve 2018 and the vacuum unit fluid supply connector 2009b, or between the air inlet valve 2018 and the wound site 2004.

FIG. 107 illustrates the treatment system 2100 schematically in more detail. The boundary or outer enclosure of the vacuum unit 2002 is illustrated by the dashed line in FIG. 107. On an upstream side of the treatment device 2003 the vacuum unit 2002 comprises the air inlet valve 2018, optionally the pressure sensor Pv and the sterile filter 2019, and on a downstream side of the treatment device 2003 the vacuum unit 2002 comprises the pump assembly 2015 and optionally a pressure sensor Pp between the pump assembly 2015 and treatment device 2003. The vacuum unit 2002 may also comprise a connection manifold 2020 providing a connection interface between the conduit 2005a, 2005b to the treatment device 2003 and the vacuum unit 2002. The connection manifold 2020 is illustrated by the dotted line in FIG. 107 and replaces connector 2009 shown in FIG. 106.

FIGS. 108 and 109 illustrate further embodiment treatment systems 2200, 2300 for supplying fluid to and removing fluid from a wound. The embodiments of FIGS. 108 and 109 include the same or similar features of the system 2100 described above with reference to FIGS. 104 to 107, however are additionally configured to provide a treatment fluid to the wound treatment device 2003.

With reference to FIGS. 108 and 109, the vacuum unit 2002 may comprise one or more ports 2025 to receive therapeutic fluids for delivery to the wound site. The port 2025 is preferably configured to be nominally closed to the passage of liquids when disconnected from the treatment fluid reservoir 2026 which subsequently opens when engaged with a leur connector. The B. Braun Medical® CARESITE™ needless connector provides an example of such a port.

A therapeutic agent in the form of a treatment fluid may be selectively delivered to the wound treatment device 2003 via the supply conduit 2012. A fluid source or treatment fluid reservoir 2026 may be coupled to the fluid port 2025 of the vacuum unit 2002, for example via a conduit or connection to an intravenous (IV) fluid giving set such as a Baxter® EMC 9608 Admin Set, B. Braun Medical® Single Chamber IV Infusion Set or similar sterile IV infusion therapy set. The treatment fluid reservoir is preferably at atmospheric pressure whilst connected to the treatment system. This can be achieved by using a non-vented IV infusion therapy set in combination with a flexible fluid bag such as Baxter® Sodium Lactate (Hartmanns or compound sodium lactate) IV Bag or similar, or it may also be achieved by connecting a vented IV infusion therapy set to a rigid or semi-rigid container of treatment fluid, such as Prontosan® Wound Irrigation Solution by B. Braun Medical®.

Example therapeutic fluids include, but are not limited to, compound sodium lactate, physiological saline (0.9% NaCL—Sodium Chloride) and 0.45% normal saline (0.45NaCL). Antimicrobial agents and solutions could also be applied for the treatment of infections and may contain agents such as polyhexanide (PHMB), silver nitrate, hypochlorous acid (HOCl), sodium hypochloride, betaine, sodium hypochlorite, super-oxidized water with neutral pH or any other antimicrobial wound irrigation solutions.

Other treatment fluids may also include cell-suspensions and cell-based fluids for promoting wound healing. The fluid to be delivered may contain one or more nutrients, ‘flowable fluids’ such as Thixotropic gels or highly viscous fluids that can still be transported via a conduit, cell-suspensions therapeutic agents for promoting wound healing. The fluid may comprise flowable gels derived from ECM, hyaluronic acid, growth factors to aid healing, to antimicrobial drugs for the treatment of infection, analgesic drugs such as fentanyl or morphine for pain relief and anti-inflammatory drugs such as ketorolac or diclofenac, for example, although other fluids are envisaged and will be apparent to a skilled person.

Instillation of autologous or allogenic cell-based therapies containing either platelet rich plasma, stem cells, stromal cells, keratinocytes, lymphocytes, bone marrow aspirate, serum and dendritic cells could aid in the repair and healing of wounds.

The instillation of chemotherapeutic drugs could also aid in the localised treatment of cancerous cells that may not be operable, or could be used as an overall treatment plan following excision of cancerous tissue.

With reference to the embodiment 2200 of FIG. 108, a treatment fluid inlet valve 2022 is selectively operable to allow fluid to flow from the treatment fluid reservoir 2026 and into the supply conduit 2012 leading to the wound. The reservoir of fluid is at atmospheric pressure. When the treatment fluid inlet valve 2022 is selectively opened, negative pressure from the pump assembly 2015 applied to the wound 2004 via the removal conduit 2011 acts to draw fluid from the treatment fluid reservoir 2026 towards the dressing or wound treatment device 2003. Upon activation of the treatment fluid inlet valve 2022 the controller (not shown in this figure) within the vacuum unit 2002 detects a subsequent drop in the vacuum pressure level at the Pv and/or Pp pressure sensor(s) and activates the pump assembly 2015 to maintain the vacuum pressure at a target level of vacuum pressure. A control algorithm is described in more detail below. In the illustrated embodiment, the air inlet valve 2018 and sterile filter 2019 is provided upstream of the therapeutic fluid valve 2022.

In the embodiment 2300 of FIG. 109, the system is without a treatment fluid inlet valve 2022. The system 2300 may include an orifice or other flow restriction to control an amount of treatment fluid introduced to the system during negative pressure treatment. In one embodiment the administration of treatment fluids is controlled via the use of an intravenous (IV) fluid giving set such as a Baxter® EMC 9608 Admin Set, B. Braun Medical® Single Chamber IV Infusion Set or similar sterile IV infusion therapy set which is connected to the unit 2002 via the fluid port 2025. The fluid flow rate of treatment fluid being introduced to the supply conduit 2012 is controlled via a roller clamp in the set, which is adjusted to vary the flow restriction within the section of tube that interfaces with the roller clamp component. In this embodiment the rate of fluid instillation can be visually checked via the drip chamber of the IV infusion set when the chamber is orientated upright, with any further flow adjustments made via the roller clamp adjustment. This embodiment provides a manual means to introduce a treatment fluid to the wound 2004 via the wound treatment device 2003.

In an alternative embodiment the vacuum unit 2002 may be connected to an infusion pump via the fluid port 2025 to allow fluids to be supplied to the wound treatment device 2003 in a selectable and controllable manner. Such infusion pump systems could include the B. Braun Medical® Vista® basic large volume infusion pump or the BD® Alaris® Syringe Module for example, which can controllably deliver from 0.1 ml/hour to 1200 ml/hour of treatment fluid on either an intermittent or constant fluid delivery basis. These systems typically offer the means to select the amount, flow rate and frequency of which treatment fluid is dispensed. When treatment fluid is introduced into the vacuum unit 2002 the system detects the subsequent drop in the set vacuum pressure level at the Pv and/or Pp pressure sensor(s) and activates the pump assembly 2015 to maintain the systems target level of vacuum pressure. A control algorithm is described in more detail below.

In the embodiments of FIGS. 108 and 109, the vacuum unit 2002 comprises a connection manifold 2021 providing a connection interface between the conduit 2005a, 2005b to the treatment device 2003 and the vacuum unit 2002 and between the vacuum unit 2002 and the treatment fluid reservoir 2026 via the fluid port 2025. The connection manifold 2021 is illustrated by the dotted line in FIGS. 108 and 109 and replaces connector 2009 shown in FIG. 106. The manifold is described in more detail below.

As described, the treatment system 2100, 2200, 2300 comprises a reservoir 2006 for collecting liquids removed from the wound site 2004, for example, wound exudate. In a preferred embodiment, the reservoir 2006 is positioned at the furthermost position away from the wound and therefore is downstream of the pump assembly 2015, for collecting fluids removed from the wound after they have passed through the pump assembly 2015. In the embodiments shown, the reservoir 2006 comprises a flexible bag. Alternatively, a rigid reservoir could be provided.

The reservoir 2006 comprises one or more air permeable filters or vents 2006a provided in a wall of the reservoir, for example a hydrophobic venting membrane provided over an aperture in the impermeable membrane. The air-permeable filter(s) or vents(s) allow the venting of gases and thereby prevent pressure build-up in the reservoir preventing effective pumping. An example reservoir has eight vents 2006a each having an 8 mm diameter and a pore size of 3 micron to sustain a high level of airflow passing through the system.

Blood clots, fibrin and other solidified fluids or tissue debris may block the venting membranes which causes the bag to inflate with air introduced to the fluid path. This inflation can cause the bag to pop and leak fluid or can prohibit the pump from generating vacuum pressure required by forcing the outlet valves from opening under excess positive pressure.

To avoid these issues a high salt compatible sodium polyacrylate polymer, or other equivalent blood compatible superabsorbent polymers may be added to the reservoir to solidify the blood and wound fluid in the bag. These polymers are available either as loose particles, particles suspended within a dissolvable PVA film pouch or polymer suspended within a textile/fabric like medium. The use of this polymer in tandem with one or more vents on the bag avoids bag inflation and allows the fluid path of the treatment system to cope with much more air as it is introduced into the system.

The pump assembly 2015 includes an inlet and outlet and is driven by a motor. In one embodiment, the pump assembly 2015 may be substantially as described in PCT/NZ2021/050205, comprising a swash plate a plurality of flexible chambers (diaphragms), a plurality of pairs of flexible valves, each pair of valves being in fluid communication with a respective flexible chamber, and a pump inlet and outlet.

The pump assembly 2015 comprises a fluid flow path through the pump from the pump inlet to the pump outlet. In a preferred embodiment the exudate reservoir 2006 is downstream of the pump assembly 2015. This means fluid from the wound passes through the pump assembly 2015.

The pump assembly 2015 preferably comprises a high capacity pump configured to maintain a negative pressure while introducing significant volumes of air to the treatment system 2100, 2200, 2300 with the air inlet valve 2018 open for a significant time portion of a valve open and close cycle time. A large capacity pump assembly 2015 is required to move the increased amount of air and lift fluid from the wound 2004 to the exudate reservoir 2006 while continuing to maintain a negative pressure at the wound 2004 at an effective negative treatment pressure level.

The air inlet valve 2018 may include an actuator such as a solenoid in electrical communication with the controller to drive the valve between open and closed positions.

The air inlet valve 2018 does not operate as a pressure relief valve, i.e. the air inlet valve is not controlled to ‘crack open’ to limit a pressure at the wound. The air inlet valve is opened and closed based on a predetermined time period, i.e. the control of the air inlet valve is temporal control, not pressure control, as explained in more detail below.

The fluid inlet valve 2022 may include an actuator such as a solenoid in electrical communication with the controller to drive the valve between open and closed positions.

Dual lumen conduits may be provided for connecting between the vacuum unit 2002 and the treatment device 2003. The conduit may have a circular outer wall.

### System Operation

In some embodiments, the pump 2015 is operated to provide a cyclical negative pressure to the wound dressing as described in U.S. application 63/117,995 incorporated herein by reference.

Preferably the controller operates the system by opening and closing the air inlet valve periodically for predetermined time periods, in a predetermined manner. This method may reduce a density of fluid at the wound to displace the fluid from the wound against gravity.

In one operating mode, in a first step, the controller (not shown) is configured to open the air inlet valve to obtain a first, low vacuum pressure at the wound treatment device and introduce air into the wound treatment device via the inlet 51, 11. Preferably this first vacuum pressure is held substantially constant, and such that the system is in an equilibrium state with a zero or constant pressure differential across the treatment device.

During the first step, the air inlet valve is preferably open for a sufficient time period so that a volume of air delivered through the system is at least a substantial portion of a total volume of the wound treatment space to displace a substantial portion of the free volume of the system. That is, enough air is supplied to move most of the fluid out of the wound treatment space.

In a second step, the controller (not shown) is configured to close the air inlet valve and operate the pump to maintain a second, higher vacuum pressure at the wound treatment device 1 . . . 1301 increasing the vacuum pressure applied to the wound and causing the wound site to contract. Preferably the first vacuum pressure is a lower vacuum pressure than the second vacuum pressure. Preferably the second vacuum pressure is held substantially constant, and such that the system is in an equilibrium state with a zero or constant pressure differential across the treatment device.

The first vacuum pressure may be about 5% to 100% of the second vacuum pressure and/or the first vacuum pressure is about 10 to 190 mmHg less than the second pressure. In an example, the first vacuum pressure is about 10 to 100 mmHg and the second vacuum pressure is about 100 to 200 mmHg.

In a second, alternate operating mode, the system is configured to administer dynamic negative pressure wound therapy without introducing a flow rate of air into the system that generates a bubble flow or slug flow comprising bubbles or slugs of air entrained in fluid flow from the wound treatment device. In a first step, the controller (not shown) is configured to open the air inlet valve 2018 to obtain a first pressure at the wound treatment device, by introducing air into the wound treatment device 1 . . . 1301 via the inlet 51, 11. During this first step, the air inlet valve 2018 is held open with the pump maintained in a stationary (not moving) state until the first pressure is reached.

In a second step of the second mode, the controller (not shown) is configured to close the air inlet valve 2018 and hold the system at the first pressure for a predetermined period. Once this time has elapsed the controller (not shown) is configured to operate the pump to return the system to a second, higher vacuum pressure at the wound treatment device 1 . . . 1301, increasing the vacuum pressure applied to the wound and causing the wound site to contract. The first vacuum pressure is a lower vacuum pressure than the second vacuum pressure.

In this second step, the vacuum pressure may be held substantially constant or the pressure may be variable (e.g. cyclic or intermittent). In an example, the first vacuum pressure is about 0 to 50 mmHg and the second vacuum pressure is about 100 to 200 mmHg. For both modes, the controller is configured to repeat the first and second steps to cycle the air inlet valve between the open and closed positions. The first and second pressures, and the predetermined hold period for the second step may be the same for every cycle, or may vary from one cycle to the next in a pre-determined way.

This second mode may be used in a continuous pattern or it may be used for a period of time to increase blood flow to the wound site, before shifting to another mode of operation, for example a constant pressure state. The second mode may be helpful to increase the rate of which granulation tissue forms in the wound by stimulating blood flow to the wound to accelerate healing.

The system may comprise various pressure sensors to assist with control of the pump based on pressure measurements. For example, a downstream pressure sensor may be located downstream of the wound treatment device and in communication with the controller, and the controller may operate the pump to achieve the vacuum pressure threshold based on a pressure sensed by the downstream pressure sensor.

Additionally or alternatively, an upstream pressure sensor may be located upstream of the wound treatment device and in communication with the controller, and the controller is configured to, operate the pump to achieve the vacuum pressure threshold based on a pressure sensed by the upstream pressure sensor. The upstream pressure sensor may be located upstream of an inlet restriction so that the upstream pressure sensor measures ambient pressure when the air inlet valve is open. The selection of mode 1 or mode 2 may be made automatically by the controller based on the pressure sensor measurements across the wound site. Alternatively, the operating mode could be selected and input by an operator such as a medical professional.

Referring again to FIGS. 108 and 109, in which the vacuum unit 2002 is connected to a source of therapeutic fluid 2026 and a wound exudate reservoir 2006 via respective conduits, and to the treatment device 2003 via a dual lumen conduit 2005. In some embodiments, the wound treatment system 2200, 2300 may not include the therapeutic fluid supply 2026.

Such systems may be configured to periodically open the air inlet valve 2018 and operate the pump 2015 to introduce filtered air into the external wound treatment device to achieve a first vacuum pressure level in the absence of a therapeutic fluid supply. The user interface 2014 of the vacuum unit 2002 may be optionally configured to provide an adjustment means, such as a button and/or other suitable user input, and a corresponding indicator such as a graphical scale and/or LED indicator light that allows the user to adjust the air inlet valve open time to compensate for the level of exudate produced for any given wound and corresponding dressing size.

Exudate produced and therefore, opening times for the air inlet valve 2018 may vary depending on the wound size, type, or healing progress. For example, a small wound requiring a 10 cm×10 cm dressing to cover the wound area with a low amount of exudate may be expected to produce approximately 30 mL of wound exudate in a day¹.

If a low profile non-adherent dressing system, such as those disclosed herein, is applied to the wound with a total dressing height of 5 mm, the volume occupied by wound therapy space 43 will be approximately 50 millilitres (50 mL), yielding a total system volume of 70 mLs for this example.

In one embodiment, the vacuum unit 2002 of is configured to supply the pump assembly 2015 with 3.3V. This yields a 178 mL/min free flow rate of air, and an air inlet valve cycle time of at least 23.6 seconds is required to supply the required 70 mL or 70 cm³ volume of filtered air calculated for the example above, to displace the fluid from the system during a single cycle through the Airflow state.

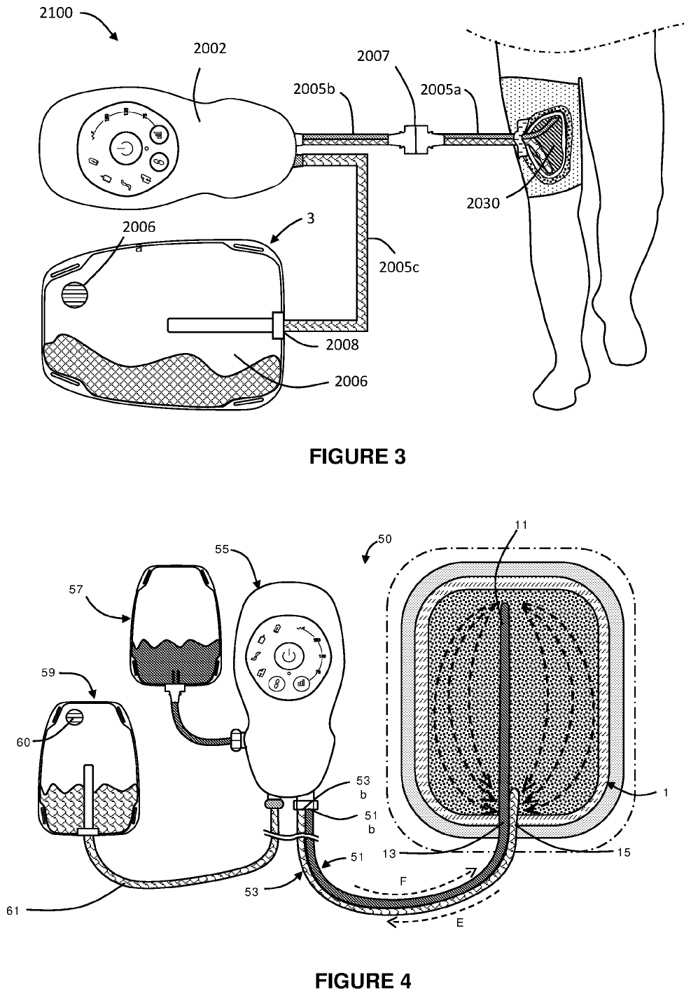

In contrast to the invention described herein, if the wound was treated using a prior art wound treatment dressing 101 as shown in FIGS. 1 to 3, such as one comprising an open cell reticulated polyurethane foam component (e.g. Granufoam®) as the porting layer to treat the 10 cm×10 cm wound described above, the volume occupied by the porting layer of the treatment device will be approximately 78.7 cm3 for the same size wound as above, yielding a total system volume of 98.7 cm³ (comprised of 78.7 cm³ of foam+20 cm³ of conduit). Granufoam™ PU foam material has been found to contract from 100 mm×104 mm×25 mm to 82 mm×96 mm×10 mm when the wound treatment space is subjected to −150 mmHg of vacuum pressure. This would require a valve open time of approximately 33.3 seconds (˜about 10 seconds longer).

In a further example, a larger wound requiring a 25 cm×25 cm dressing to cover the wound and with a high amount of exudate may be expected to produce approximately 1,750 mL in a day¹. If the same vacuum unit 2002 of the embodiment system and low profile non-adherent dressing system, such as those disclosed herein, (as described above) is applied to the wound, a total system volume of 332.5 mL requires an air inlet valve cycle time of at least 112 seconds to supply the 332 cm³ (332 mL) volume of filtered air required to displace the fluid from the during a single cycle through the Airflow state.

In this example it may also be advantageous to provide a user interface 2014 that provides the user with an option to increase the frequency airflow cycles in a given day to manage the high level of exudate in the wound, where this example would require at least 6 cycles within a 24 hour period to cope with the 1,750 millilitres of exudate produced.

In some systems the vacuum unit 2002 may be further connected to a source of therapeutic fluid 2026 as described previously for embodiments 2200 and 2300 of FIGS. 108 and 109. In such embodiments, the user interface 2014 may provide input means to enable a user to adjust the dispensed volume of fluid to compensate for the total system volume of the wound treatment system 2004.

In one such embodiment, the user interface 2014 of the vacuum unit 2002 could provide a means for setting the volume of the dressing in a separate adjustment to that of the level of exudate produced at the wound. The user interface 2014 may include a button that allows a user to set the free volume of the system, for example, by pressing and holding a button to draw fluid through the system at a set vacuum pressure level, such as 30 mmHg. The set vacuum pressure level for introducing and holding fluid within the system could be set anywhere from 10 mmHg to 200 mmHg, but most preferably is between 10 mmHg and 125 mmHg.

The user interface 2014 of the vacuum unit 2002 may additionally provide a means to adjust a dwell time for any instilled fluid to be held within the treatment device 2040. The hold time may be specified as any time period but most preferably is for a duration of between 1 minute to 30 minutes. The pump unit 2 may additionally include a means to oscillate the vacuum pressure level from the first fluid instillation pressure level to a second pressure level, including the duration of time spent at a first pressure level and second pressure level.

Other variables that may provide useful to adjust via the user interface 2014 of the vacuum unit include the operating mode of the pump switching between an oscillating pressure mode to a continuous supplied vacuum pressure mode, or adjusting the time elapsed at each vacuum pressure level.

Other variables that may provide useful for adjustment will be known to those trained in the art.

When the therapy is complete, to remove the dressing, the occlusive layer is peeled off the skin, and the conduit and wound therapy device are removed. The vacuum pump may comprise a reservoir for collecting exudate liquids removed from the dressing.

In a final step, if used, the mouldable seal can be removed from the patient's skin by stretching the mouldable seal in a longitudinal direction or removing with a solvent, as described in PCT application PCT/NZ2020/050044 incorporated herein by reference. As the seal is elongated, it gently releases from the patient's skin minimising the likelihood of damage to the skin.

If used, the bioresorbable layer does not need to be removed from the wound, as it degrades naturally with time.

The dressings described above, and other embodiments thereof are intended for use in the treatment of chronic wounds, for example diabetic ulcers and burns.

### REFERENCES

1. Malmsjo, M., Huddleston, E., & Martin, R. (2014). Biological effects of a disposable, canisterless negative pressure wound therapy system. Eplasty, 14.

1. A wound interface device for use in negative pressure wound therapy, the device comprising:
a flexible body having a plurality of spacers, the spacers configured to define a therapy space between a wound surface and a wound facing surface of the device body;
an inlet for the instillation of fluids to a wound site; and
an outlet for the removal of fluids from the therapy space;
wherein the inlet is configured to deliver fluid to an area adjacent a first end of the device, and the outlet is configured to remove fluid from an area adjacent to a second, opposite, end of the device.

2. (canceled)

3. A wound interface device as claimed in claim 1, wherein the spacers define multiplicity of channels between the first and second ends of the device;
wherein the channels are shaped to direct fluids laterally across the wound surface between the first and second ends of the device.

4. (canceled)

5. A wound interface device as claimed in claim 1, wherein the spacers are shaped and distributed to prevent collapse of the spacers and/or of the therapy space under the application of negative pressure to the therapy space.

6. A wound interface device as claimed in claim 1, wherein the spacers comprise shaped projections.

7. A wound interface device as claimed in claim 6, wherein the spacers each comprise a convex wound contacting surface.

8. A wound interface device as claimed in claim 1, wherein the body comprises a compliant, flexible member that flexes to conform to the contours of various wound surfaces.

9. A wound interface device as claimed in claim 1, that is trimmable to fit within the perimeter of various wounds.

10. A wound interface device as claimed in claim 1, wherein the inlet comprises an inlet port for coupling to a fluid source; and an inlet channel in fluid communication with the inlet port, configured to deliver fluid to a first end of the device.

11. A wound interface device as claimed in claim 10, wherein the outlet comprises an outlet port for coupling to a negative pressure source; and an outlet channel in fluid communication with the outlet port, configured to remove fluid from a second end of the device.

12. A wound interface device as claimed in claim 11, wherein the inlet channel extends in a first direction, and the outlet channel extends in a second direction that is perpendicular to the first direction.

13. A wound interface device as claimed in claim 12, wherein the outlet channel extends in a transverse direction of the device.

14. A wound interface device as claimed in claim 12, wherein the inlet and outlet ports are provided on a moulded member extending from an opposite side of the device body to the wound facing side.

15. A wound interface device as claimed in claim 11, wherein the outlet channel comprises an elongate slot positioned to allow the ingress of fluids from the therapy space into the outlet channel.

16. A wound interface device as claimed in claim 15, wherein the slot extends in a transverse direction of the device.

17. A wound interface device as claimed in claim 11, wherein the outlet channel comprises a plurality of apertures to allow the ingress of fluids from the therapy space into the outlet channel.

18. A wound interface device as claimed in claim 17, wherein the outlet channel apertures comprise slits that are substantially perpendicular to a plane of the body of the device.

19. A wound interface device as claimed in claim 1, wherein the body is formed from an elastomeric material.

20. A wound interface device as claimed in claim 1, wherein the body is formed from silicone.

21. A wound interface device as claimed in claim 1, wherein the body is liquid and air impermeable.

22. A wound dressing for applying negative pressure to a wound, the dressing comprising:
a wound interface device as claimed in claim 1; and
a liquid impermeable occlusive outer layer.

23. A wound dressing as claimed in claim 22, further comprising a bioresorbable layer for placing between the wound interface device and the wound surface.

24. A wound dressing as claimed in claim 23, wherein the bioresorbable layer comprises a plurality of apertures or slits to enable fluid flow from the wound to the porting layer.

25. A wound dressing as claimed in claim 24, wherein the apertures or slits are substantially X-shaped, Y-shaped, C-shaped, U-shaped, or V-shaped.

26. A wound dressing as claimed in claim 23, wherein the bioresorbable layer comprises a multiplicity of small pieces of bioresorbable material.

27. A wound dressing as claimed in claim 23, wherein the bioresorbable layer comprises extracellular matrix (ECM).

28. A wound dressing as claimed in claim 22, wherein the wound dressing comprises an inlet port and an outlet port, and comprising an inlet conduit for coupling to the inlet port for the supply of fluids to the wound and an outlet conduit for the application of negative pressure to the wound.

29. A wound dressing as claimed in claim 28 wherein the inlet and outlet conduits are provided by a dual lumen conduit.

30. A wound dressing as claimed in 29, wherein the dual lumen conduit comprises a primary conduit to couple to the outlet to apply a negative pressure to the dressing and a secondary conduit to couple to the inlet for introducing fluid to the dressing and/or for facilitating pressure measurement.

31. A wound dressing as claimed in claim 22, wherein the occlusive layer comprises a polyurethane sheet comprising an adhesive surface.

32. A wound dressing as claimed in claim 22, wherein the wound dressing comprises a mouldable adhesive seal for surrounding a wound, wherein the seal comprises butyl rubber, a filler, and a tackifying resin.

33. A wound dressing as claimed in claim 32, wherein the seal is removable and re-sealable against a patient's skin.

34. A wound dressing as claimed in claim 32, wherein the seal is non-curing.

35. A mouldable and removable adhesive seal for surrounding a wound, the seal comprising butyl rubber, a filler, and a tackifying resin.

36. A kit of parts comprising the wound interface device as claimed in claim 1; and a liquid impermeable occlusive outer layer.

37. A kit of parts as claimed in claim 36, further comprising a mouldable adhesive seal.

38. A kit of parts as claimed in claim 37, wherein a first removable release sheet is adhered to one side of the mouldable adhesive seal, and a second removable release sheet adhered to a second side of the mouldable adhesive seal, wherein the second removable release sheet is stretchable.

39. A kit of parts as claimed in claim 36, further comprising a connector for coupling to the inlet and outlet ports of the wound interface device.

40. A kit of parts as claimed in claim 39, wherein the connector is a two-part connector.

41. A kit of parts as claimed in claim 39, wherein the wound interface device comprises a boss having a chamfered surface, and wherein the connector comprises a complementary chamfered surface for sealingly engaging with the chamfered surface of the boss.

42. A wound therapy system comprising a wound dressing as claimed in claim 22, and a pump coupled to the device outlet and configured to apply a negative pressure to the wound dressing.

43. A wound therapy system as claimed in claim 42, wherein the occlusive layer is adhered to a patient's skin and over the wound therapy device.

44. A wound therapy system as claimed in claim 42, further comprising a reservoir for collecting exudate removed from the dressing.

45. A wound therapy system as claimed in claim 42, further comprising a reservoir for storing treatment fluids for administering to the wound.

46. A wound therapy system as claimed in claim 42, wherein the pump is coupled to the wound dressing by way of a dual lumen conduit.

47-72. (canceled)

73. A wound interface device for use in negative pressure wound therapy, the device comprising:
a flexible body having a plurality of projections configured to define a therapy space between a wound surface and a wound facing surface of the device body and to conform to the contours of various wound surfaces; and
an outlet for the application of negative pressure and removal of fluids from the therapy space.
wherein the projections define multiplicity of channels between the first and second ends of the device, the channels shaped to direct fluids laterally across the wound surface between the first and second ends.

74. A wound interface device for use in negative pressure wound therapy, the device comprising:
a flexible body having a plurality of shaped protrusions each comprising a convex wound contacting surface configured to define a therapy space between a wound surface and a wound facing surface of the device body, the protrusions being shaped and distributed to prevent collapse of the spacers and/or of the therapy space under the application of negative pressure to the therapy space;
an outlet for the application of negative pressure and removal of fluids from the therapy space; and
a transverse outlet channel comprising a plurality of apertures to allow the ingress of fluids from the therapy space into the outlet channel.
